(19) United States
(12) Patent Application Publication
(10) Pub. No.: US 2009/0227572 A1
(43) Pub. Date: Sep. 10, 2009

(54) TREATMENT OF DIABETIC WOUNDS AND PERIPHERAL NEUROPATHIES

(75) Inventors: Jack R. Barber, San Diego, CA (US); Shi Chung Ng, San Diego, CA (US)

Correspondence Address: ROPES & GRAY LLP, PATENT DOCKETING 39/361, 1211 AVENUE OF THE AMERICAS, NEW YORK, NY, 10036-8704, US

(73) Assignee: Cytrx Corporation, Los Angeles, CA (US)

(21) Appl. No.: 12/405,915

(22) Filed: Mar. 17, 2009

Related U.S. Application Data
(63) Continuation-in-part of application No. PCT/US08/05794, filed on May 5, 2008.
(60) Provisional application No. 60/927,603, filed on May 4, 2007.

(51) Int. Cl. A61K 31/5395; A61K 31/4545; A61K 31/15; C07D 413/14; C07D 401/12; C07C 251/16

(52) U.S. Cl. 514/229.2; 514/318; 514/640; 544/66; 546/193; 564/265

(57) ABSTRACT

The present invention provides methods of enhancing healing of wound associated with diabetes, comprising administering an effective amount of one or more of certain hydroxylamine derivatives to a subject in need thereof. In another aspect, the instant invention provides methods of treating or preventing peripheral nervous system neuropathies. Peripheral nervous system neuropathies may but need not be diabetic neuropathies, and may but need not be associated with a diabetic wound. The invention also provides pharmaceutical compositions comprising a certain hydroxylamine derivative or a pharmaceutically acceptable salt thereof, optionally in combination with one or more additional therapeutic agents. In certain compositions and methods, the additional therapeutic agent is a second hydroxylamine derivative or a pharmaceutically acceptable salt thereof.

This application is a continuation-in-part of and claims the benefit of International Application No. PCT/US2008/005794, filed May 5, 2008, which claims the benefit of U.S. Provisional Application No. 60/927,603, filed May 4, 2007, the specification of each of which is incorporated by reference herein in its entirety. International Application No. PCT/US2008/005794 was published under PCT Article 21(2) in English.

### BACKGROUND OF THE INVENTION

Diabetes affects 18.2 million people, 6.3% of the US population, with 1.3 million new cases diagnosed each year. In addition to the pathological metabolic condition, diabetes is one of the leading causes of non-trauma induced amputation due to poor wound healing.

The effects of diabetes on healing are diverse, multifactorial, complex and inter-related, and the underlying mechanisms of the impairment are poorly understood. In diabetes, many of the processes in the phases of wound healing, including inflammation, granulation tissue formation, re-epithelialization, angiogenesis, and lymphangiogenesis, are impaired. (Falanga, V, Lancet 2005, 366:1736-1743). Another factor that contributes to the poor wound healing in diabetic patients is impaired circulation. In model mice subjects in which type 1 diabetes is simulated by treating mice with streptozotocin (STZ), pressure induced vasodilation is impaired. Thus, endothelial dysfunction is also counted among the factors underlying the formation and/or impaired healing of diabetic ulcers. (Sigaudo-Roussel et al., Diabetes 53: 1564-1569, 2004). Further, impaired microcirculation, partially caused by inadequate angiogenesis compared with that which occurs in a normal wound healing process, may contribute to the poor wound healing. Perhaps for that reason, growth factors and agents that promote angiogenesis have been reported to assist diabetic wound healing. For example, Saaristo et al., Am. J. Pathol. 2006, 169:1080-1087 report that vascular endothelial growth factor improved wound healing in a diabetic mouse model system.

Diabetic wound is also characterized by impaired inflammatory cell function, decreased secretion of cytokines/growth factors, and a prolonged inflammatory phase. (Wetzler, C. et al., J Invest. Dermatol. 2000, 115:245-253).

Another facet of diabetic wound such as foot ulcer is its neuropathy. These tissues lack normal innervation, and there has been evidence that, separate from the direct effect of diabetes, denervated tissues suffer from impaired wound healing (Smith, P. G. and Liu, M., Cell Tissue Res. 2002 March; 307 (3):281-91 Epub 2002 Feb. 5). Treatment with growth factors such as nerve growth factors have been suggested as a way to accelerate wound healing.

While some of these biologics may be helpful in treating diabetic patients, biologics are not without problems with regard to, for example, stability, batch-to-batch consistency of preparation, or availability in large quantities. There is therefore a need for small molecules that are useful in enhancing wound healing. Further, as the causal relationships of the multiple symptoms that accompany diabetes, including the impaired wound healing, are still unknown and whether alleviating one factor would be noticeably significant to the wound healing as a whole is unknown, a method of treatment to enhance wound healing is much desired.

### SUMMARY OF THE INVENTION

The instant invention provides methods of enhancing healing of wound accompanying diabetes, comprising administering an effective amount of one or more of certain hydroxylamine derivatives to a subject in need thereof, i.e., a subject that has developed wound, for example foot ulcer, in conjunction with diabetes. In another aspect, the instant invention provides methods of treating or preventing peripheral nervous neuropathies, such as a diabetic neuropathy, for example, associated with a diabetic wound. In certain embodiments, the diabetic neuropathy is not associated with a diabetic wound. In other embodiments, the peripheral nervous system neuropathy is not diabetic neuropathy, for example, a peripheral neuropathy associated with chemotherapy. Certain of the hydroxylamine derivatives useful for practicing the methods of the invention include, but are not necessarily limited to, those previously described in U.S. Pat. No. 5,147,879; U.S. Pat. No. 6,143,741; U.S. Pat. No. 6,653,326; U.S. Pat. No. 6,649,628; U.S. Pat. No. 6,384,029; U.S. Pat. No. 5,328,906; U.S. Pat. No. 5,296,606; U.S. Pat. No. 5,919,796; U.S. Pat. No. 6,002,002; U.S. Pat. No. 6,180,787; U.S. Pat. No. 6,384,029; and U.S. Pub. 2005/0043295.

Preferred compounds for use in the methods of the invention are:
N-[2-hydroxy-3-(1-piperidinyl)propoxy]-3 pyridine-carboximidoyl-chloride (bimoclomol),

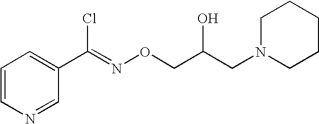

N-[2-hydroxy-3-(1-piperidinyl)propoxy]-3-pyridine-1-oxide-3-carboximidoyl chloride (arimoclomol),

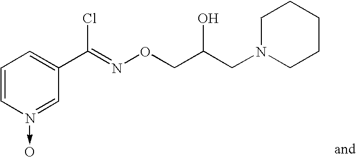

5,6-dihydro-5(1-piperidinyl)-methyl-3-(3-pyridyl)-4H-1,2,4-oxadiazine (iroxanadine),

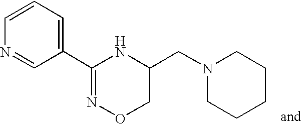

N-[3-(1,1-dimethylethyl)amino]2-hydroxypropoxy]-3-trifluoromethylbenzene-carboximidoyl chloride (Compound 1).

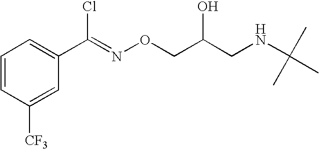

The structure of any of the above compounds is intended to include all stereochemical forms of the compound, including geometric isomers (i.e., E, Z) and optically active isomers (i.e., R, S). Single stereochemical isomers as well as enantiomeric and diastereomeric mixtures of the present compounds are within the scope of the invention. Unless otherwise stated, formulae depicted below are also meant to include compounds which differ only in the presence of one or more isotopically enriched atoms, and all pharmaceutically acceptable salts of any of the foregoing.

Any of the above compounds may be used alone or in combination, and optionally in combination with one or more additional therapeutic agents for the treatment of diabetes and pathological conditions that occur associated with diabetes, such as diabetic neuropathy. In certain embodiments, one or more of the above compounds may also be used alone or in combination for the treatment or prevention of peripheral nervous system neuropathies, such as a diabetic neuropathy, for example, associated with a diabetic wound. In certain embodiments, the diabetic neuropathy is not associated with a diabetic wound. In other embodiments, the peripheral nervous system neuropathy that is not diabetic neuropathy, for example, a neuropathy associated with chemotherapy. Preferred additional therapeutic agents are provided.

More generally, an embodiment of the method of the present invention may also be carried out using pharmaceutical compositions comprising a compound of Formula (I) or Formula (II):

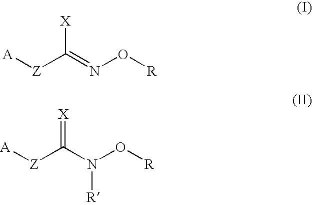

pharmaceutically acceptable salts thereof or hydrates thereof, wherein, in each of compounds of Formulae (I) and (II):

A is an alkyl, substituted alkyl aralkyl, aralkyl substituted in the aryl and/or in the alkyl moiety, aryl, substituted aryl, heteroaryl or substituted heteroaryl group;

Z is a covalent bond, oxygen or NR³;

R³ is selected from the group consisting of hydrogen, an alkyl substituted alkyl aryl, substituted aryl, aralkyl, or aralkyl substituted in the aryl and/or in the alkyl moiety;

R is an alkyl or substituted alkyl,

X, in compound of Formula (I), is halogen or a substituted hydroxy or amino, monosubstituted amino or disubstituted amino group and, in compound of Formula (II), is oxygen, imino or substituted imino group;

R′ is hydrogen, an alkyl, substituted alkyl, aryl, substituted aryl, aralkyl, aralkyl having substituted aryl and/or alkyl moiety, acyl or substituted acyl group;

In certain embodiments, the methods of the invention comprise administering one or more additional therapeutic agents in combination with one or more hydroxylamine derivatives. In a preferred embodiment, the method comprises administering the combination of arimoclomol and iroxanadine. In another embodiment, the additional therapeutic agent is a drug that alleviates symptoms associated with diabetes or a drug that treats or prevents complications arising from diabetic wounds. In another embodiment, the additional therapeutic agent is a drug that alleviates symptoms associated with peripheral nervous system neuropathies, such as a diabetic neuropathy, for example, associated with a diabetic wound. In certain embodiments, the diabetic neuropathy is not associated with a diabetic wound. In other embodiments, the peripheral nervous system neuropathy is not diabetic neuropathy, for example, a neuropathy associated with chemotherapy. In particular embodiments, the additional therapeutic agent is selected from anti-inflammatory, antibiotics, antifungal, antiviral, growth factors, hormones and neuroprotective agents.

The invention also provides pharmaceutical compositions comprising one or more hydroxylamine derivatives of the invention for the treatment of diabetic wounds and optionally, comprising an additional therapeutic agent or agents. In another embodiment, the invention also provides pharmaceutical compositions comprising one or more hydroxylamine derivatives for the treatment or prevention of peripheral nervous system neuropathies, such as a diabetic neuropathy, for example, associated with a diabetic wound. In certain embodiments, the diabetic neuropathy is not associated with a diabetic wound. In other embodiments, the peripheral nervous system neuropathy is not diabetic neuropathy, for example, a neuropathy associated with chemotherapy.

According to certain preferred embodiments, the pharmaceutical compositions of this invention are administered orally, topically, parenterally, peritoneally, intravaginally, intrarectally, or by any other desired route of administration under the therapeutic circumstances that is known and accepted to those of skill in the art.

### BRIEF DESCRIPTION OF THE DRAWINGS

FIG. 1A-B shows the weight change of mice over the experimental time course. Panel A shows the data for homozygous diabetic mice, and Panel B shows the data for heterozygous, asymptomatic mice.

FIG. 2A-F shows the assessment of wound healing when either iroxanadine (Groups 1, 4-8), arimoclomol (Groups 2, 9), or no drug (Group 3) was administered. Panel A shows the assessment of healing by comparing the percentage closure of the perimeter for homozygous mice, and Panels B and C show the ANOVA analysis results of Day 14 for the homozygous mice (Global p=0.003), Panel B graphically, and C numerically. Panels D and E show the results for heterozygous (non-diabetic) control groups, Panel D graphically and E numerically. Panel F shows the assessment of healing by comparing the percentage closure of the area of the wound.

FIG. 3A-I shows and relates to Kaplan-Meier curves showing proportion of mice with closed wounds. Panel A compares heterozygous control and treated; Panel B compares heterozygous and homozygous controls; Panels C-G compare homozygous groups treated with various iroxanadine concentrations to control; and Panel H compares a homozygous group treated with arimoclomol at various concentrations to control. Panel I is a numerical representation of the results.

FIG. 4A-C shows the hazard ratio of time for wounds to close on each mouse, i.e., the likelihood of wound closure (for both wounds to close on each mouse). Panels A and B show the data for both wounds to close, Panel A graphically and Panel B numerically and Panel C shows the data for the first wound.

FIG. 5A-F shows the time to closure of wound. Panels A and B show the median time for the closure of each wound, analyzed by a non-parametric analysis, Panel A graphically and Panel B numerically. Panels C and D show the mean time for the closure of both wounds, analyzed by the t-test analysis, Panel C graphically and Panel D numerically. Panel E shows the rate of wound in all groups analyzed as two groups, 0-8 days and 9-12 days. The treatment during 0-8 days included ethanol in the samples, and the treatment during 9-12 days were free of ethanol. Panel F shows the median and mean time to closure of each wound as Box and Whisker plots, and the legend for the graphic representation.

FIG. 6 shows the wound healing data for mice that were systemically administered iroxanadine, 10 mg/kg IP, b.i.d. compared to control vehicle over time (0-20 days).

FIG. 7 shows the wound healing data for mice that received topical administration of arimoclomol (4% w/v aqueous solution, with carboxy methyl cellulose).

FIG. 8 shows body weights of the experimental animals described in Example 3.

FIG. 9 shows the dose-response effect on the glycemia levels at day 7 and day 41 with administration of iroxanadine to STZ diabetic rats, as described in Example 3.

FIG. 10 shows a dose response effect on the SNCV performance 3 days before STZ induced diabetes (day −3 on graph) and on days 25 and 39 post-STZ induced diabetes with administration of iroxanadine, as described in Example 3.

FIG. 11 shows a dose response effect on the CMAP latency performance 3 days before STZ induced diabetes (day −3 on graph) and on days 25 and 39 post-STZ induced diabetes with administration of iroxanadine, as described in Example 3.

FIG. 12 shows the effect on IENF density of post-STZ rats as described in Example 3.

FIG. 13 shows photomicrographs (×100) of the IENFs in the different experimental groups of the rat paw skin biopsy.

### DETAILED DESCRIPTION OF THE INVENTION

### 1. Definitions

For convenience, certain terms employed in the specification, examples, and appended embodiments, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “prophylactic” or “therapeutic” treatment refers to administration to a subject of one or more of the compositions of the invention. If it is administered prior to clinical manifestation of the unwanted condition (e.g., disease or other unwanted state of the host animal) then the treatment is prophylactic, i.e., it contributes to prevention of, i.e., protection of the host against developing the unwanted condition, whereas if administered after manifestation of the unwanted condition, the treatment is therapeutic (i.e., it is intended to diminish, ameliorate or prevent progression of the unwanted condition or side effects therefrom).

The term “therapeutic effect” refers to a local or systemic effect in animals, particularly mammals, and more particularly humans, caused by a pharmacologically active substance or substances. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain compounds discovered by the methods of the present invention may be administered in a sufficient amount to produce a reasonable benefit/risk ratio applicable to such treatment.

The term “effective amount” refers to the amount of a therapeutic reagent that when administered to a subject by an appropriate dose and regimen produces at least one desired result.

A “subject” or “patient” to be treated by the method according to the invention can mean either a human or non-human animal, preferably a mammal.

The term “subject in need of treatment for a disorder” is a subject diagnosed with that disorder, demonstrating symptoms or surrogate markers associated with the disorder, or is suspected of having that disorder.

Throughout this specification, the word “comprise” or variations such as “comprises” or “comprising” will be understood to imply the inclusion of a stated integer or groups of integers but not the exclusion of any other integer or group of integers.

The term “alkyl” refers to straight or branched, saturated aliphatic hydrocarbon containing 1 to 21 carbon atoms. “Short chain alkyl” refers to an alkyl group containing from 1 to 8 carbon atoms. Examples of short chain alkyl groups include, but are not limited to, methyl, ethyl, propyl, isopropyl, butyl, isobutyl, sec-butyl, pentyl, tert-pentyl, hexyl, heptyl, and octyl groups. Preferably, the short chain alkyl contains from 1 to 6 carbon atoms and is selected from the group consisting of methyl, ethyl, propyl, isopropyl, butyl, isobutyl, sec-butyl, pentyl, tert-pentyl, and hexyl-groups. “Long chain alkyl” refers to an alkyl group containing from 9 to 21 carbon atoms. Examples of long chain alkyl groups include, but are not limited to, nonyl, decyl, undecyl, dodecyl, tridecyl, tetradecyl, pentadecyl, hexadecyl, heptadecyl, octadecyl, nonadecyl, eicosyl and heneicosyl groups. Preferably the long chain alkyl contains from 9 to 17 carbon atoms and is selected from the group consisting of nonyl, decyl, undecyl, dodecyl, tridecyl, tetradecyl, pentadecyl, hexadecyl, and heptadecyl groups.

The term “cycloalkyl” refers to a monocyclic, non-aromatic, hydrocarbon ring system containing 3 to 8 carbon atoms. “Short cycloalkyl chain” refers to a cycloalkyl group containing from 3 to 8 carbon atoms. Examples include, but are not limited to, cyclopropyl, cyclobutyl, cyclopentyl, cyclohexyl, cycloheptyl and cyclooctyl groups. Preferably, the cycloalkyl group contains from 3 to 7 carbon atoms and is selected from the group consisting of cyclopropyl, cyclobutyl, cyclopentyl, cyclohexyl and cycloheptyl.

The term “aryl” refers to a mono- or polycyclic ring system which contains 6, 10, 12 or 14 carbons in which at least one ring of the ring system is aromatic. Examples of aryl ring systems include, but are not limited to, phenyl, naphthyl, pentalenyl, anthracenyl groups. Preferably, the aryl group is phenyl or naphthyl groups.

The term “aralkyl” refers to an alkyl group, wherein one or more hydrogen atoms of the alkyl group is replaced by one or more aryl radical. Examples of aralkyl groups include, but are not limited to, benzyl, benzhydryl, trityl, 1-phenyl-ethyl, 2-phenylethyl, 2-benzhydryl-ethyl, 3-phenylpropyl, 1-methyl-2-phenyl-ethyl, 1-phenylbutyl, 4-tritylbutyl, 1,1-dimethyl-2-phenylethyl, 4-phenylbutyl, 5-phenylpentyl, and 6-phenylhexyl-groups. Preferably, the aralkyl group is a lower alkyl group containing from 1 to 4 carbon atoms, substituted with a phenyl group. Preferred aralkyl groups include, but are not limited to, benzyl, 1-phenylethyl, 2-phenylethyl, and 1-methyl-2-phenylethyl groups.

The term “heterocyclic” refers to a mono ring system which contains 1 to 15 carbon atoms and 1 to 4 heteroatoms, in which the ring system may optionally contain unsaturated bonds but is not aromatic. Heteroatoms are independently sulfur, nitrogen, or oxygen. Examples include, but are not limited to, aziridinyl-, azetidinyl-, oxaziranyl-, pyrrolidinyl-, imidazolidinyl-, pyrazolidinyl-, perhydro-thiazolyl-, perhydro-isoxazolyl-, piperidinyl-, piperazinyl-, perhydro-pyrimidinyl-, perhydro-pyridazinyl-, morpholinyl-, perhydro-1H-azepinyl, oxazolyl, and isoxazolyl, oxadiazolyl (e.g. 1,2,4-oxadiazolyl- and others). Preferably, the heterocyclic ring is a 3-8 membered ring system. More preferably, the heterocyclic ring is a 5-8 membered ring system. More preferably, the heterocyclic ring is 5-6 membered ring, containing 1-2 oxygen atoms and 1-3 N-atoms.

The term “heteroaryl” refers to a mono- or polycyclic ring system which contains 1 to 15 carbon atoms and 1 to 4 heteroatoms, and in which at least one of the rings in the ring system is aromatic. Heteroatoms are sulfur, nitrogen or oxygen. Preferably, the heteroaryl group is an unsaturated, 3-8 membered ring. More preferably, the heteroaryl group is a 5-6 membered, 1-4 N-containing unsaturated hetero-monocyclic group. Examples include, but are not limited to, pyrrolyl, pyrrolinyl, imidazolyl, pyrazolyl, pyridyl group and its N-oxide, pyrimidinyl, pyrazinyl, pyridazinyl, triazolyl, tetrazolyl, and dihydrotriazinyl. Preferably, the heteroaryl group is a polycyclic ring containing 1-5 N-atoms. Examples include, but are not limited to, indolyl, isoindolyl, indolizinyl, benzimidazolyl, quinolyl, isoquinolyl, indazolyl, benzotriazolyl, tetrazolopyridyl, tetrazolopyridazinyl, and dihydro-triazolopyridazinyl. Preferably, the heteroaryl group is a polycyclic ring containing an unsaturated ring, 1-2 oxygen atoms and 1-3 N-atoms. Examples include, but are not limited to, benzoxazolyl and benzoxadiazolyl. Preferably, the heteroaryl group is a monocyclic, 3-8 membered ring, more preferably 5-6 membered ring, containing 1-2 sulfur atoms and 1-3 N-atoms. Examples include, but are not limited to, thiazolyl, 1,2-thiazolyl, thiazolinyl, and thiadiazolyl. Preferably, the heteroaryl group is a monocyclic, 3-8 membered ring, more preferably 5-6 membered ring, containing one sulfur atom or one oxygen atom. Examples include, but are not limited to, thienyl and furanyl. Preferably, the heteroaryl is a bicyclic ring containing 1-2 sulfur atoms and 1-3 nitrogen atoms. Examples include, but are not limited to, benzothiazolyl and benzothiadiazolyl.

The term “acyl” group refers to an acyl group which might be a short chain alkanoyl (e.g., formyl, acetyl, propionyl, butyryl and the like), a short chain alkoxy-carbonyl (e.g., methoxy-carbonyl, ethoxy-carbonyl, propoxy-carbonyl, butoxy-carbonyl, tert-butoxy-carbonyl and the like), a short chain alkyl-sulphonyl (e.g., methyl-sulphonyl, ethyl-sulphonyl and the like), aryl-sulphonyl (e.g., phenyl-sulphonyl and the like), aroyl (e.g., benzoyl, naphthoyl and the like), aryl-(short chain alkanoyl) (e.g., phenyl-acetyl, phenyl-propionyl and the like), cyclo-(short chain alkyl)-(short chain alkanoyl) (e.g., cyclohexyl-acetyl and the like), aryl-(short chain alkoxy)-carbonyl (e.g., benzyloxy-carbonyl and the like), aryl-carbamoyl (e.g., phenyl-carbamoyl, naphthyl carbamoyl and the like), cycloalkyl-carbamoyl (e.g., cyclohexyl-carbamoyl and the like), hetero-monocyclic sulphonyl (e.g., thienyl-sulphonyl, furyl-sulphonyl and the like). Acyl group may be optionally substituted with 1-3 substituents as described above.

The term “terminal amino-alkyl” group refers to a short chain alkyl group containing a substituted N-atom in the terminal position of the alkyl chain and in which the alkyl chain is optionally substituted with one or more substituents, preferably with one or two halogen (e.g., chloro, bromo, fluoro, iodo), hydroxyl group or acylated hydroxyl group. Preferably, one or two short chain alkyl groups and the “alkyl” definition is the same as written above. The N-atom in the ω-position of the alkyl chain can be substituted with one or two short chain alkyl substituents, preferably methyl-, ethyl-, tert-butyl- and the like, with cycloalkyl carbamoyl- (e.g., cyclohexyl-carbamoyl- and the like). Preferably, the N-atom can be a part of a saturated heterocyclic group which contains 1-4 nitrogen atoms and is selected from the group consisting of aziridinyl, azetidinyl, oxaziranyl, pyrrolidinyl, imidazolidinyl, pyrazolidinyl, perhydro-thiazolyl, perhydro-isoxazolyl, piperidinyl, piperazinyl, perhydro-pyrimidinyl, perhydro-pyridazinyl, morpholinyl, and perhydro-1H-azepinyl. The N-atom in the ω-position can be substituted with an aryl group (e.g., phenyl and the like), and can be quaternarized by a short chain alkyl substituent or oxidized as well.

The term “halogen” refers to F, Cl, Br, or I.

The term “optionally substituted” aryl or alkyl refers to an aryl- or alkyl group having one or more substituents. Examples of substituents include, but are not limited to, cyano, hydroxyl, short chain alkyl (e.g., methyl, ethyl, propyl, isopropyl, butyl, isobutyl, sec-butyl, pentyl, tert-pentyl, hexyl, heptyl, octyl and the like), short chain alkoxy (e.g., methoxy, ethoxy, propoxy, isopropoxy, butoxy, isobutoxy, sec-butoxy, tert-butoxy, pentyloxy, tert-pentyloxy, hexyloxy and the like), aryl (e.g., phenyl, naphthyl, and the like), nitro, amino, mono-(short chain alkyl)-substituted amino (e.g., methyl, ethyl, propyl, isopropyl, tert-butyl)-amino and the like, di-(short chain alkyl)-substituted amino (e.g., dimethylamino, diethylamino, dipropylamino, diisopropylamino, dibutylamino, dipentylamino, dihexylamino and the like), monohalogen, dihalogen or trihalogen (short chain)-alkyl (e.g., chloromethyl, 2,2-dichloroethyl, trifluoromethyl and the like) or halogen atom (e.g. fluoro-, chloro-, bromo-, and iodine atom).

The term “bioavailable” means that at least some amount of a particular compound is present in the systemic circulation. Formal calculations of oral bioavailability are described in terms of an F value (“Fundamentals of Clinical Pharmacokinetics,” John G. Wegner, Drug Intelligence Publications; Hamilton, Ill. 1975). F values are derived from the ratio of the concentration of the parent drug in the systemic circulation (e.g., plasma) following intravenous administration to the concentration of the parent drug in the systemic circulation after administration by a non-intravenous route (e.g., oral). Therefore, oral bioavailability within the scope of the present invention contemplates the ratio or F value of the amount of parent drug detectable in the plasma after oral administration compared to intravenous administration.

The term “treating” or “treatment” is intended to mean mitigating or alleviating at least on symptom of a condition, disease or disorder in a mammal, such as a human, or the improvement of an ascertainable measurement associated with a condition, disease or disorder.

The term “pharmaceutically acceptable derivative” refers to any pharmaceutically acceptable salt, ester, or salt of such ester, of a compound of this invention or any other related compound which, upon administration to a recipient, is capable of providing (directly or indirectly) a compound of this invention or a metabolite or residue thereof.

### 2. Embodiments

The instant invention relate to methods of enhancing healing of wound accompanying diabetes, comprising administering an effective amount of one or more of certain hydroxylamine derivatives to a subject in need thereof, i.e. a subject that has developed wound, for example foot ulcer, in conjunction with diabetes. The instant invention also relates to methods of treating or preventing peripheral nervous system neuropathies, comprising administering an effective amount of one or more of the hydroxylamine derivatives to a subject in need thereof, for example, to a subject that may develop or that has developed said peripheral nervous system neuropathy. In some embodiments, the peripheral nervous system neuropathy is a diabetic neuropathy, for example, associated with a diabetic wound. In certain embodiments, the diabetic neuropathy is not associated with a diabetic wound. In other embodiments, the peripheral nervous system neuropathy is not diabetic neuropathy, for example, a neuropathy associated with chemotherapy. Hydroxylamine derivatives useful for practicing the methods of the invention include e.g., those previously described in:

U.S. Pat. No. 5,147,879; U.S. Pat. No. 6,143,741; U.S. Pat. No. 6,653,326; U.S. Pat. No. 6,649,628; U.S. Pat. No. 6,384,029; U.S. Pat. No. 5,328,906; U.S. Pat. No. 5,296,606; U.S. Pat. No. 5,919,796; U.S. Pat. No. 6,002,002 U.S. Pat. No. 6,180,787; U.S. Pat. No. 6,384,029; and U.S. Pub. 2005/0043295.

In, one embodiment, the methods of the invention comprise the step of administering N-[2-hydroxy-3-(1-piperidinyl)propoxy]-3 pyridine-carboximidoyl-chloride (bimoclomol):

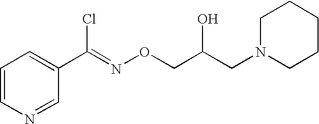

Bimoclomol was described in U.S. Pat. No. 5,147,879, and may be prepared by methods well known to those skilled in the art for analogous compounds. In particular, see U.S. Pat. No. 6,180,787, which is incorporated herein by reference. Bimoclomol is a racemic mixture.

In another embodiment, methods of the invention comprise the step of administering N-[2-hydroxy-3-(1-piperidinyl)propoxy]-pyridine-1-oxide-3-carboximidoyl chloride (arimoclomol),

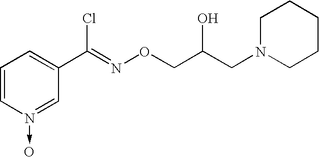

As described elsewhere, arimoclomol may be used to treat a patient suffering from diabetes. In particular, the present invention describes the use of arimoclomol for enhancing wound healing.

Arimoclomol may be prepared by methods well known to those skilled in the art for analogous compounds. See, e.g., U.S. Pat. No. 6,649,628 and PCT Publication WO 01/79174, (which are incorporated by reference herein). Arimoclomol is a R(+) enantiomer.

Yet another embodiment of the invention utilizes 5,6-dihydro-5(1-piperidinyl)-methyl-3-(3-pyridyl)-4H-1,2,4-oxadiazine (iroxanadine).

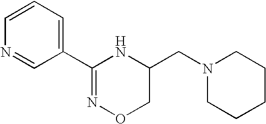

Iroxanadine and related compounds were previously described in PCT Publication WO 98/06400 and U.S. Pat. No. 6,384,029 (which are incorporated herein by reference), and may be prepared by methods well known to those skilled in the art for analogous compounds, e.g., as described in these publications. Iroxanadine was previously recognized by its effect on endothelial cell protection from stress, especially in the reoxygenation phase following ischemia. Kabakov et al., Cell. Mol. Life. Sci. 61 (2004) 3076-3086.

Another compound useful for practicing the methods of invention is N-[3-(1,1-dimethylethyl)amino-2-hydroxypropoxy]-3-trifluoromethylbenzene-carboximidoyl chloride (Compound 1)

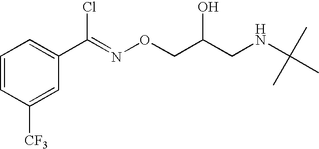

Compound 1 may be prepared by methods well known to those skilled in the art for analogous compounds. See, e.g., U.S. Pat. No. 6,649,628 and PCT Publication WO 01/79174, which are incorporated by reference herein. Compound 1 may be prepared, for example, using methods described for the preparation of arimoclomol in the above references, e.g., by starting with CF₃-cyanobenzene instead of CN-pyridine and substituting piperidine with tert-butylamine.

These compounds have previously been described as enhancing endothelial cell protection (iroxanadine) or alleviating diabetic and other neuropathies (arimoclomol). The novel use of these compounds for enhancing wound healing in a subject afflicted by diabetes is now described as one embodiment of the instant invention.

In particular embodiments, arimoclomol, iroxanadine, and Compound 1, alone or in combination with each other or with other therapeutic agents, are found to be effective in the enhancement of diabetic wound healing. In preferred embodiments, isolated arimoclomol, iroxanadine, or Compound 1, alone or in combination with each other or with other therapeutic agents, is administered for enhancing wound healing to a subject afflicted with diabetes.

In certain embodiments, arimoclomol, iroxanadine, and Compound 1, either alone or in combination with each other or with one or more other therapeutic agents, are found to be effective in the treatment or prevention of peripheral nervous system neuropathies. In certain embodiments, the peripheral nervous system neuropathy is diabetic neuropathy. In some embodiments, the diabetic neuropathy is associated with a diabetic wound. In other embodiments, the diabetic neuropathy is not associated with a diabetic wound.

In some embodiments, arimoclomol, iroxanadine, and Compound 1, alone or in combination with each other or with other therapeutic agents, are found to be effective in the treatment or prevention of peripheral nervous system neuropathies excluding diabetic neuropathy. In certain embodiments, the peripheral nervous system neuropathy excluding diabetic neuropathy is neuropathy associated with chemotherapy.

More generally, certain embodiments of the present invention may be carried out using pharmaceutical compositions comprising a compound of Formula (I) or Formula (II):

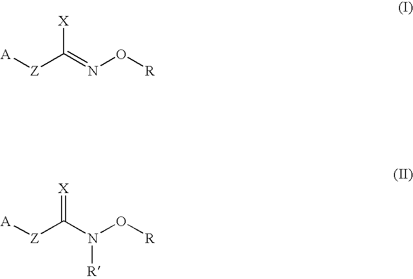

and pharmaceutically acceptable salts thereof, wherein, in each of compounds of Formulae (I) and (II):

A is an alkyl, substituted alkyl aralkyl, aralkyl substituted in the aryl and/or in the alkyl moiety, aryl, substituted aryl, heteroaryl or substituted heteroaryl group;

Z is a covalent bond, oxygen or NR³;

R³ is selected from the group consisting of hydrogen, an alkyl substituted alkyl aryl, substituted aryl, aralkyl, or aralkyl substituted in the aryl and/or in the alkyl moiety;

R is an alkyl or substituted alkyl,

X, in compound of Formula (I), is halogen or a substituted hydroxy or amino, monosubstituted amino or disubstituted amino group and, in compound of Formula (II), is oxygen, imino or substituted imino group; and

R′ is hydrogen, an alkyl, substituted alkyl, aryl, substituted aryl, aralkyl, aralkyl having substituted aryl and/or alkyl moiety, acyl or substituted acyl group.

The formula for any of the above compound is intended to include all stereochemical forms of the compound, including geometric isomers (i.e., E, Z) and optically active isomers (i.e., R, S). Single stereochemical isomers as well as enantiomeric and diastereomeric mixtures of the present compounds are within the scope of the invention. Unless otherwise stated, formulae depicted herein are also meant to include compounds which differ only in the presence of one or more isotopically enriched atoms. For example, compounds having the present formulae except for the replacement of a hydrogen by a deuterium or tritium, or the replacement of a carbon by a ¹³C— or ¹⁴C-enriched carbon are within the scope of this invention.

In some embodiments, the compound of Formula (I) or (II) has the “R” configuration at the carbon that is directly attached to the hydroxyl group. In some embodiments, the compound of Formula (I) or (II) has the “S” configuration at the carbon that is directly attached to the hydroxyl group.

In some embodiments, the compound of Formula (I) or (II) has the “E” configuration across the carbon-nitrogen double bond. In some embodiments, the compound of Formula (I) or (II) has the “Z” configuration across the carbon-nitrogen double bond.

In one embodiment, in compounds of Formula (I), Z is a covalent bond and X is a halogen. In some aspects of this embodiment, X is chloro or bromo. In some aspects of this embodiment, A is selected from the group consisting of (i) aralkyl or aralkyl having substituted aryl moiety; (ii) aryl or substituted aryl; (iii) naphthyl; (iv) an N-containing heteroaryl group, including those which may be condensed with a benzene ring; (v) an S-containing heteroaryl group and (vi) an O-containing heteroaryl group. In some aspects of this embodiment, A is phenyl alkyl or phenyl alkyl having one or more substituents, preferably alkoxy. In other aspects of this embodiment, A is phenyl or substituted phenyl. In some aspects of this embodiment, A is substituted phenyl containing one or more substituents selected from the group consisting of alkyl, halo, haloalkyl, alkoxy and nitro. In some aspects of this embodiments, A is pyridyl. In further aspects of this embodiment, R is selected from the group consisting of (i) a terminal amino-alkyl, (ii) a terminal amino-alkyl having mono or disubstituted amino moiety; (iii) a terminal amino alkyl having substituted alkyl moiety; (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety.

Compounds of Formula (I) in which Z is a covalent bond and X is a halogen are disclosed in U.S. Pat. Nos. 5,147,879, 5,328,906, and 5,296,606, each of which is incorporated herein by reference. These compounds can be prepared by procedures described in the cited U.S. patents, preferably by diazotization of the corresponding derivatives (when X is NH₂) in the presence of the appropriate hydrohalide. The starting compounds can be obtained by known procedures, e.g., those described in Hungarian Patent No. 177.578 (1976), namely by coupling an amidoxime of Formula (1) (R¹═R²═H):

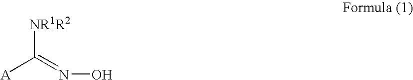

with e.g. a reactive derivative of Formula (2):

R-L Formula (2)

in the presence of a base, and can be diazotized usually without isolation or purification. The terminal groups A and R of the compounds can be further amidified or derivatized, as desired.

In another embodiment, in compounds of Formula (I), Z is covalent bond and X is a substituted hydroxy group O-Q, wherein Q is an unsubstituted or substituted alkyl or aralkyl group. In one aspect of this embodiment, Q is a straight or branched alkyl. In one aspect of this embodiment, A is aryl or heteroaryl; and R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, A is a N-containing heteroaromatic group. In some aspects of this embodiment, when R is (iv), the alkyl group is substituted with a hydroxy or acyloxy group. In some aspects of this embodiment, any of the terminal amino-alkyl groups (i)-(iv) is a 3-8 carbon atom alkyl moiety.

In another embodiment, in the compound of Formula (I), Z is a covalent bond, X is O-Q, Z is a covalent bond, and R is a—CH₂—CH(OH)—R″. The compound is cyclized through the hydroxy group and is represented by Formula (I′):

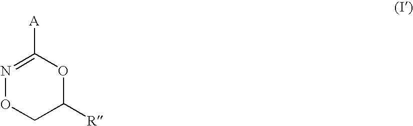

R″ is selected from the group consisting a straight or branched alkyl and a substituted straight or branched alkyl. In some aspects of this embodiment, R″ is a terminal amino-alkyl which is optionally substituted on its amino group. In some aspects of this embodiment, R″ is a terminal amino-alkyl which is substituted on its amino group with a C₁₋₅ straight or branched alkyl chain. In some aspects, R″ is a terminal amino-alkyl mono- or disubstituted on the amino group, wherein the amino-substituents, independently from each other may be one or two straight or branched alkyl or cycloalkyl, or the two amino-substituents, together with the adjacent N-atom form a 3- to 7-membered heterocyclic ring. In some aspects, the heterocyclic ring is a 5- to 7-membered, optionally containing an additional heteroatom. In some aspects, A is selected from the group consisting of phenyl, substituted phenyl, N-containing heteroaryl, substituted N-containing heteroaryl, S-containing heteroaryl, and substituted S-containing heteroaryl.

Compounds of Formula (I′) in which Z is a covalent bond and X is a O-Q are disclosed in Hungarian Patent Application No. 2385/1992, which is incorporated by reference. These compounds may be prepared from compounds of Formula (I) in which Z is covalent bond and X is halogen by procedures described in the Hungarian. Pat. Appln. No. 2385/1992, e.g., by reaction with alkoxides, or by alkaline ring closure for the cyclic compounds of Formula (I′).

In another embodiment, in the compounds of Formula (I), Z is a covalent bond and X is NR₁R₂, wherein R₁ and R₂ are independently selected from the group consisting of H, straight or branched alkyl, substituted straight or branched alkyl, cycloalkyl, or R₁ and R₂, together with the nitrogen atom to which they are bound, form a saturated 3- to 7 membered heterocyclic ring. In some aspects of this embodiment, R₁ and R₂ form a saturated 5- to 7 membered heterocyclic ring. In some aspects of this embodiment, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety. In some aspects of this embodiment, A is selected from the group consisting of (i) aralkyl or aralkyl having substituted aryl moiety; (ii) aryl or substituted aryl; (iii) naphthyl; (iv) an N-containing heteroaryl group, including those which may be condensed with a benzene ring; (v) an S-containing heteroaryl group and (vi) an O-containing heteroaryl group. In some aspects of this embodiment, A is phenylalkyl or substituted phenylalkyl having one or more substituents. In some aspects of this embodiment, A is phenyl alkyl substituted by one or more alkoxy groups. In some aspects of this embodiment, A is phenyl or substituted phenyl. In some aspects of this embodiment, A is substituted phenyl containing one or more substituents selected from the group consisting of alkyl, halogen, haloalkyl, alkoxy, nitro, and acylamino group. In other aspects of this embodiment, A is pyridyl.

Compounds of Formula (I) in which Z is a covalent bond and X is NR¹R² are disclosed in Hungarian Patent No. 177578 (1976) and U.S. Pat. No. 6,653,326, each of which is incorporated herein by reference. These compounds may be synthesized by alkylation of unsubstituted amidoxime derivatives of compounds of Formula (I) (Formula (I) wherein R¹═R²═H) with a reactive derivative of compounds of Formula (II) in presence of a base.

In another embodiment, in the compound of Formula (I), Z is a covalent bond, X is NR₁R₂, and R is a—CH₂—CH(OH)—R″. The compound is cyclized through the NR₁R₂ group and is represented by Formula (I″):

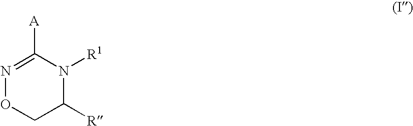

wherein R″ is selected from the group consisting of straight or branched alkyl or a substituted straight or branched alkyl. R¹ is selected from the group consisting of hydrogen, unsubstituted or substituted straight or branched alkyl, cycloalkyl, unsubstituted aralkyl and aralkyl substituted in the aryl- and alkyl moiety. In some aspects of this embodiment, A is selected from the group consisting of (i) aryl or substituted aryl; (ii) naphthyl; (iii) an N-containing heteroaryl group, including those which may be condensed with a benzene ring; (iv) S-containing heteroaryl group; and (v) O-containing heteroaryl group. In some aspects, A is phenyl or substituted phenyl. In some aspects, A is substituted phenyl containing one or more of alkyl, halogen, haloalkyl, alkoxy, amino or nitro group. In further aspects, R″ is selected from the group consisting of (i) a terminal amino-alkyl having mono or disubstituted amino moiety and (ii) a terminal amino alkyl having mono or disubstituted amino moiety and also substituted alkyl moiety. In some aspects of this embodiment, any of the terminal amino-alkyl groups (i) or (ii) is a 3-8 carbon atom alkyl moiety. In some aspects, the terminal amino-alkyl group (i) or (ii) has disubstituted amino moiety, wherein the substituents, together with the nitrogen atom attached thereto, form a saturated 3- to 7 membered heterocyclic ring. In some aspects, the heterocyclic ring is 5- to 7 membered and optionally contains an additional heteroatom. In some aspects, in the terminal amino-alkyl groups (i) or (ii) the amino-substituent is a straight or branched alkyl group or cycloalkyl.

Compounds of Formula (I″) may be prepared by ring closure between atoms N(4)-C(5) using the open chain compound of Formula (I) in which Z is a covalent bond, X is ═NR¹R², wherein R¹ is as defined in connection with the compounds of the Formula (I″) above, R² is H, R is —CH₂—CHY⁵—R″, where Y⁵ is a leaving group, e.g., a halogen atom. Such derivatives may be obtained from the corresponding Y⁵═OH compounds with inorganic halogenating agents, e.g., thionyl chloride or phosphorus pentachloride. The halogenation may be carried out with or without an inert solvent, e.g. benzene, chloroform, tetrahydroturane etc., usually by boiling. After removing the excess of the reagent, e.g., by evaporation of the thionyl chloride, the crude halogen derivative may be cyclized—either after or with-out isolation or purification—by treatment with a strong base, e.g., potassium butoxide in t-butanol to give compound of Formula (I″), which is finally isolated and purified by standard procedures (extraction, recrystallization, etc).

According to one embodiment, in the compound of Formula (I), Z is oxygen and X is O-Q, wherein Q is selected from the group consisting of alkyl substituted alkyl, aralkyl, and substituted aralkyl having substituted aryl or substituted alkyl moiety. In some aspects of this embodiment, when A is alkyl or substituted alkyl, it contains 1-4 carbon atoms. In some aspects, A is selected from the group consisting of a C₁₋₄ alkyl or substituted alkyl aralkyl and substituted aralkyl having substituted aryl or substituted alkyl moiety. In some aspects of this embodiment, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group.

The compounds of Formula (I) in which Z is oxygen and X is O-Q may be prepared by the reaction of O-substituted hydroxylamines of Formula (6): (see e.g., Ger. Off. 2,651,083 (1976)) and orthoesters of Formula (7):

H₂N—O—R Formula (6)

C(OQ)₄ Formula (7)

The condensation may be carried out in the regent itself, as a solvent, preferably by boiling. After evaporation, the product may be isolated by crystallization, if there is an amine function in the side chain R, in the form of acid addition salt.

According to one embodiment, in the compound of Formula (I), Z is oxygen, X is NR¹R², and R¹ and R² are independently selected from the group consisting of H, a straight or branched alkyl a substituted straight or branched alkyl cycloalkyl, aryl, and substituted aryl, or R¹ and R², together with the nitrogen atom attached thereto, form a 3- to 7 member saturated heterocyclic ring. In some aspects, R¹ and R² form a 5- to 7 membered saturated heterocyclic ring. In some aspects of this embodiment, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety. In some aspects of this embodiment, A is selected from the group consisting of (i) alkyl or substituted alkyl; (iii) aralkyl or aralkyl having substituted aryl and/or substituted alkyl moiety; and (iv) aryl or substituted aryl. In some aspects of this embodiment, A is phenyl or substituted phenyl.

The compounds of Formula (I) may be prepared as described hereinbelow, wherein the methods depend on the nature of X, namely whether X is an unsubstituted amino (NH₂) or a substituted amino functionality.

Compounds of Formula (I) in which X is NH₂ may be prepared by the addition of hydroxylamine of Formula (6) to an organic cyanate of formula A-O—CN (see, e.g., Chem. Ber. 98, 144 (1965)). The reaction may carried out preferably in an inert organic solvent, usually at room temperature. The isolation often requires chromatographic purification.

Compounds of Formula (I) in which X is monosubstituted amino group (e.g., NHR¹) may be prepared from known haloformimidates of Formula (9):

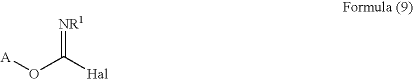

(see, e.g., Houben-Weil, “Methoden der Organischen Chemie,” Band E/4, p. 544 (1983) and a compound of Formula (6) in the presence of an organic base (e.g., triethylamine) or an inorganic base, such as sodium carbonate in an inert solvent, as benzene, tetrahydroturane, etc., followed by standard work-up and purification procedures.

Compounds of Formula (I) in which X is a disubstituted amino group may be prepared by the reaction of a secondary amine of Formula 5 with a compound of Formula (I), where Z is oxygen and X is O-Q (which may be prepared by the method described above):

HNR¹R² Formula (5)

These amination reactions are performed in polar organic solvents, e.g., ethanol, by refluxing, if necessary.

According to another embodiment, in the compound of Formula (I), Z is NR³, wherein R³ is selected from the group consisting of hydrogen, an alkyl, substituted alkyl, aryl, substituted aryl, aralkyl, and aralkyl having substituted aryl or substituted alkyl moiety; and X is NR¹R², wherein R¹ and R² independently selected from the group consisting of H, a straight or branched alkyl a substituted straight of branched alkyl aryl or substituted aryl, cycloalkyl, and R¹ and R², together with the nitrogen atom attached thereto, form a saturated 3- to 7 membered heterocyclic ring.

In some aspects of this embodiment, A is selected from the group consisting of alkyl, substituted alkyl, aralkyl, aralkyl having substituted aryl or substituted alkyl moiety, aryl, and substituted aryl group. In some aspects, R¹ and R² form a saturated 5- to 7 membered heterocyclic ring. In further aspects of this embodiment, R is selected from the group consisting of (i) a terminal amino-alkyl, (ii) a terminal-amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety.

Compounds of Formula (I) in which Z is NR³ and X is NR¹R², may be prepared by aminolysis of the corresponding isourea derivatives belonging to a group of compounds described above (i.e., compounds of Formula (I) in which Z is oxygen and X is NR¹R²) with ammonia or a primary or secondary amine. The reaction may be carried out preferably in a polar solvent, e.g., water or ethanol, using excess of the amine. Alternatively, haloformamides of Formula (10) (Houben-Weil “Methoden der Organischen Chemie,” Band E/4, page 553 (1983)) may be reacted with a compound having Formula (6) in the presence of an organic or inorganic base to give compounds of this group as well:

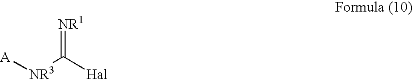

The reaction may be carried out in inert organic solvent, usually at ambient temperature.

Compounds of Formula (I) in which R is a group of the Formula (b):

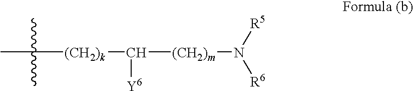

wherein R is acyl, may be prepared by esterifying the corresponding compounds containing hydrogen as R⁷. The alkyl or aryl esters may be obtained by using an acid chloride or anhydride in the presence of a tertiary amine or an inorganic base, preferably in an inert solvent.

It should be understood, however, that the group of compounds described above excludes hydroxylamine derivatives of the following structure:

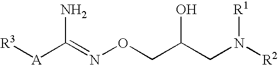

wherein

R¹ is H or C₁₋₅ alkyl,

R² is H, C₁₋₅ alkyl C₃₋₈ cycloalkyl or phenyl which may be substituted with OH or phenyl, R¹ and R², when taken together with the adjacent nitrogen atom, form a 5-8 membered saturated or unsaturated ring, which optionally contains one or more additional N, O or S atom(s) and may be condensed with a benzene ring,

R³ is H or phenyl, naphthyl, or pyridyl optionally substituted with one or more halo or C₁₋₄ alkoxy,

A is a group of the formula (a),

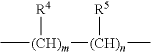

wherein

R⁴ is H or phenyl,

R⁵ is H or phenyl,

m is 0, 1 or 2, and

n is 0, 1 or 2.

According to another embodiment, the present invention provides compounds of Formula (II). In one aspect of this embodiment, in the compound of Formula (II), Z is covalent bond and X is oxygen. In further aspects of this embodiment, A is selected from the group consisting of (i) alkyl, aralkyl or aralkyl having substituted aryl or alkyl moiety; (ii) aryl or substituted aryl; (iii) an N-containing heteroaryl group; and (iv) S-containing heteroaryl group. In some aspects of this embodiment, A is phenyl or substituted phenyl having one or more substitutents. In some aspects of this embodiment, A is substituted phenyl containing one or more substituents selected from the group consisting of alkyl, haloalkyl and alkoxy. In other aspects of this embodiment, A is pyridyl.

In further aspects, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety.

In further aspects, R′ is selected from the group consisting of hydrogen, an alkyl, substituted alkyl, aryl, substituted aryl, aralkyl, and aralkyl having substituted aryl or alkyl moiety.

Compounds belonging to this group are disclosed in Hungarian Patent Application No. 2385/1992, which is incorporated herein by reference. These compounds may be prepared according to the methods described therein, most preferably, they can be obtained by acylation of O-substituted hydroxylamine derivatives having Formula (6) (see also, e.g., Ger. Off. 2,651,083 (1976)) with an acid chloride having Formula (11):

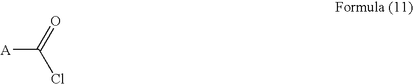

This route may also be employed for the preparation of compounds in which R′ is other than hydrogen, using a compound of Formula (12) —instead of Formula (6) —as starting material:

R¹HN—O—R Formula (12)

According to another embodiment, in compounds of Formula (II), Z is a chemical bond; X is NR⁴, wherein R⁴ is selected from the group consisting of H, an alkyl substituted alkyl, aryl, substituted aryl, aralkyl, aralkyl having substituted aryl or substituted alkyl group, cycloalkyl; and R⁴ is selected from the group consisting of alkyl, substituted alkyl, aryl, substituted aryl, aralkyl, and aralkyl having substituted aryl or substituted alkyl moiety. In some aspects of this embodiment, A is (i) aralkyl or aralkyl having substituted aryl moiety; (ii) aryl or substituted aryl; (iii) naphthyl; (iv) an N-containing heteroaryl group; and (v) S-containing heteroaryl group. In some aspects of this embodiment, A is phenyl alkyl or phenyl alkyl having one or more substituents. In some aspects of this embodiment, A is phenyl alkyl substituted by one or more alkoxy groups. In some aspects of this embodiment, A is phenyl or substituted phenyl. In some aspects of this embodiment, A is substituted phenyl containing one or more substituents selected from the group consisting of alkyl, haloalkyl and nitro. In other aspects of this embodiment, A is pyridyl.

In some embodiments, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety.

These compounds may be prepared either by O-alkylation of a N,N′-disubstituted amidoxime of Formula (13):

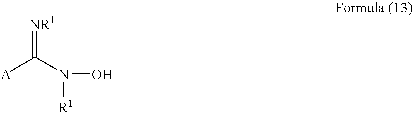

with a chemical compound having Formula (2) (for the reaction conditions, see preparation of compounds of Formula (I), wherein Z is covalent bond and X is NR¹R²), or by O-acylating an N,O-disubstituted hydroxylamine of the Formula (12) with an imidoyl halide of the Formula (16):

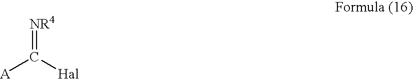

The reaction may be carried out in an inert solvent, preferably in the presence of an organic or inorganic acid scavenger.

The compounds wherein R is a group of the Formula (b)

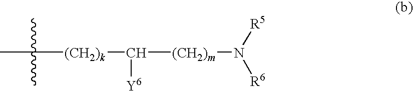

wherein R is acyl, may be prepared by esterifying the corresponding compounds containing hydrogen as R⁷. The alkyl or aryl esters may be obtained by using an acid chloride or anhydride in the presence of a tertiary amine or an inorganic base, preferably in an inert solvent.

According to one embodiment, in compounds of Formula (II), Z is oxygen and X is oxygen. In some aspects of this embodiment, A is selected from the group consisting of alkyl, substituted alkyl, aralkyl, and aralkyl with substituted aryl or alkyl moiety. In some aspects, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety A) and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety. In some aspects of this embodiment, R′ is selected from the group consisting of hydrogen, an alkyl, substituted alkyl, aryl, substituted aryl, aralkyl, and aralkyl with substituted aryl or alkyl moiety.

According to this embodiment, the compounds are disclosed in Hungarian Patent Application No. 1756/95 (filed Jun. 15, 1995), which is incorporated herein by reference. These compounds may be prepared by acylation of a hydroxylamine having, Formula (6) or Formula (12) with a chloroformate having Formula (14), in a similar manner as with the simple acid chlorides, as described for the synthesis of compounds of Formula (II) wherein Z is covalent bond and X is oxygen. The reaction requires the presence of a base, inorganic or organic, and may be performed in an inert solvent, e.g., in chloroform. The side-product salt is removed, e.g., by extraction with water, and the product is isolated from the organic solution.

In yet another embodiment, in the compounds of Formula (II), Z is oxygen; X is NR⁴, wherein R⁴ is selected from the group consisting of alkyl, substituted alkyl, aralkyl, substituted aralkyl having substituted aryl or substituted alkyl group, aryl, substituted aryl, heteroaryl and substituted heteroaryl group. In some aspects of this embodiment, A is selected from the group consisting of alkyl, substituted alkyl, aryl, substituted aryl, aralkyl and aralkyl with substituted aryl or alkyl moiety. In some aspects of this embodiment, A is an unsubstituted or substituted phenyl.

In some aspects of this embodiment, R is ω-aminoalkyl, which suitably contains a hydroxy or acyloxy group in the alkyl chain, and is optionally substituted on the amine nitrogen, wherein the alkyl chain of the ω-aminoalkyl group preferably contains 3 to 8 carbon atoms. In some aspects of this embodiment, R′ is selected from the group consisting of alkyl, aryl or aralkyl which groups may be unsubstituted or substituted.

According to this embodiment, these compounds of Formula (I), wherein Z is oxygen and X is NR¹R² may be prepared, similarly from haloformimidates having Formula (9) and a chemical compound having Formula (12), in the presence of an organic base (e.g., triethylamine) or inorganic base, e.g sodium carbonate in an inert solvent, as benzene, tetrahydrofurane etc., followed by standard work-up and purification procedures.

In another embodiment, in the compounds of Formula (II), Z is NR³, wherein R³ is selected from the group consisting of hydrogen, an alkyl, substituted alkyl, aryl, substituted aryl, aralkyl, and aralkyl having substituted aryl or substituted alkyl moiety; and X is oxygen. In some aspects of this embodiment, A is selected from the group consisting of (i) aralkyl or aralkyl having substituted alkyl or aryl moiety; (ii) aryl or substituted aryl; (iii) an N-containing heteroaryl group; (iv) an alkyl or substituted alkyl, straight or branched; and (v) a cycloalkyl group. In some aspects of this embodiment, A is phenyl alkyl or phenyl alkyl having one or more substituents. In some aspects of this embodiment, A is phenyl or substituted phenyl. In some aspects of this embodiment, A is substituted phenyl containing one or more substituents selected from the group consisting of alkyl, alkoxy, halogen, haloalkyl and nitro group. In other aspects of this embodiment, when a is (iv), the alkyl group contains 4 to 12 carbon atoms.

In some aspects of this embodiment, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety. In some aspects, R′ is selected from the group consisting of hydrogen, an alkyl, substituted alkyl, aralkyl, aralkyl having substituted aryl or alkyl moiety, aryl, substituted aryl, acyl and substituted acyl group.

According to this embodiment, these compounds are disclosed in a Hungarian Patent Application No. 1756/95, which is incorporated herein by reference, and may be prepared by the reaction of a hydroxylamine compound having Formula (6) or Formula (12) with an isocyanate having Formula (15):

A-N═C═O Formula (15)

in an inert solvent, usually by simple stirring of the mixture at room temperature for 2-24 hours. Finally, the products may be isolated, following evaporation of the solvent. In some aspects, the product may be isolated by crystallization.

In another embodiment, in the compounds of Formula (II), Z is NR³, wherein R³ is selected from the group consisting of hydrogen, an alkyl, substituted alkyl, aryl, substituted aryl, aralkyl, and aralkyl having substituted aryl or substituted alkyl moiety; X is NR⁴, wherein R⁴ is selected from the group consisting of H, an alkyl, substituted alkyl, aryl, substituted aryl, aralkyl, aralkyl having substituted aryl or substituted alkyl group, and cycloalkyl; and R′ is selected from the group consisting of alkyl, substituted alkyl, aralkyl, substituted aralkyl having substituted aryl or substituted alkyl moiety, aryl and substituted aryl. In some aspects of this embodiment, R³ is selected from the group consisting of hydrogen, alkyl and substituted alkyl; R⁴ is hydrogen or an aryl group; and A is selected from the group consisting of alkyl, substituted alkyl, aryl and substituted aryl, or aralkyl, which may be substituted in the aryl and/or alkyl moiety. In further aspects, R is selected from the group consisting of (i) a terminal amino-alkyl, (ii) a terminal amino-alkyl having mono or disubstituted amino moiety; (iii) a terminal amino alkyl having substituted alkyl moiety; and (iv) a terminal amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the terminal amino-alkyl group is a 3-8 carbon atom alkyl moiety.

According to this embodiment, the compounds may be prepared by aminolysis of the corresponding isourea derivatives (compounds of Formula (II), wherein Z is oxygen and X is NR⁴) with a primary or secondary amine or ammonia. The reaction may be carried out preferably in a polar solvent, e.g., water or ethanol, using an excess of the amine. Alternatively, the compounds may be prepared by reacting haloformamidines of Formula (10) with a compound of Formula (12) in the presence of an organic or inorganic base in inert solvents, usually at their boiling point.

According to one embodiment, the present invention provides compounds of Formula (I) in which X is halogen; Z is a chemical bond and A is a group of the Formula (a) wherein Y¹ is selected from the group consisting of halo, alkoxy, a nitro group and a haloalkyl group; and n is selected from the group consisting of 1, 2, and 3; or O-containing heteroaryl, S-containing heteroaryl, or N-containing heteroaryl group which may be condensed with a benzene ring; and R is a group having Formula (b), wherein R⁵ and R⁶, independently from each other, are selected from the group consisting of H, a straight or branched alkyl, and cycloalkyl, or R⁵ and R⁶, when taken together with the nitrogen atom attached thereto, form a saturated 3- to 7-membered heterocyclic ring; Y⁶ is —OR⁷ wherein R⁷ is H or an acyl group; k is 1, 2 or 3; and m is 1, 2, or 3, with the proviso, that when A is pyridyl or naphthyl, or a group of the Formula (a) wherein Y¹ is halo or alkoxy, then R⁷ is other than H. These compounds may optionally contain as A an N-containing heteroaromatic group with N-quaternary C₁₋₄ alkyl or the oxide of the said N-containing heteroaromatic group and/or an R wherein the ring formed by the terminal groups R⁶ and R⁷ is an N-quaternary or N-oxidized saturated heterocyclic ring.

In some aspects of this embodiment, X is chloro or bromo. In some aspects of this embodiment, Y¹ is haloalkyl containing 1-4 carbon atoms. In other aspects, Y¹ is selected from the group consisting of furyl, thienyl, pyridyl, quinolyl, and isoquinolyl. In some aspects of this embodiment, R⁵ and R⁶, independently from each other, is substituted straight or branched alkyl. In some aspects, R⁵ and R⁶ is C₁₋₄ alkyl. In other aspects, when R⁵ and R⁶ are taken together with the nitrogen atom attached thereto form a saturated 3- to 7-membered heterocyclic ring, preferably the resulting ring is a 5- to 7-membered saturated heterocyclic ring. In some aspects, R⁷ is selected from the group consisting of alkyl carbonyl, substituted alkyl carbonyl, aryl carbonyl or substituted aryl carbonyl, and aminoacyl or substituted aminoacyl.

In some aspects of this embodiment, A is a group of the Formula (a) wherein Y¹ is trifluoromethyl. In some aspects of this embodiment, X is halo, A is pyridyl, Z is a chemical bond, and R is the group of the Formula (b) wherein R⁵ and R⁶ independently from each other are selected from the group consisting of H, straight or branched alkyl, and cycloalkyl, or R⁵ and R⁶ together with the adjacent N atom form a saturated 3- to 7-membered heterocyclic ring, Y⁶ is —OR⁷ wherein R⁷ is aminoacyl, k is 1, 2 or 3 and m is 1, 2 or 3. In some aspects, R⁵ and R⁶ independently from each other are C₁₋₄ alkyl or cycloalkyl. In other aspects, R⁵ and R⁶ together with the adjacent N atom form a saturated 5- to 7-membered heterocyclic ring. According to each aspect of this embodiment, the compounds may be optically active.

In certain embodiments, a method for the treatment or prevention of a peripheral neuropathy defined as a diabetic neuropathy associated with a diabetic wound excludes compounds of Formula (I), wherein R is a group of formula (d), unsubstituted by Y³, X is halo, Z is a chemical bond, and A is defined as pyridyl or N-oxy pyridyl.

In other embodiments, a method for the treatment or prevention of a peripheral neuropathy defined as a diabetic neuropathy associated with a diabetic wound excludes compounds of Formula (I), wherein Z is a chemical bond, X is halo, A is a group of formula (c), and R is a group of formula (d), wherein k and m are both 1.

In certain embodiments, a method for the treatment or prevention of a peripheral neuropathy defined as a diabetic neuropathy excludes compounds of Formula (I), wherein R is a group of formula (d), unsubstituted by Y³, X is halo, Z is a chemical bond, and A is defined as pyridyl or N-oxy pyridyl.

In other embodiments, a method for the treatment or prevention of a peripheral neuropathy defined as a diabetic neuropathy excludes compounds of Formula (I), wherein Z is a chemical bond, X is halo, A is a group of formula (c), and R is a group of formula (d), wherein k and m are both 1.

According to this embodiment, these compounds may be prepared using procedures that are analogous to those described in U.S. Pat. Nos. 5,147,879: 5,398,906; and 5,996,606, each of which is incorporated herein by reference. For example, compounds in which both R⁵ and R⁶ are other than hydrogen, may be prepared by the diazotization of the corresponding NH₂ derivatives (i.e., the compound of Formula (I) in which Z is covalent bond and X is NH₂) in the presence of the appropriate hydrogen halide, similarly to the procedure described in U.S. Pat. Nos. 5,147,879; 5,328,906, and 5,296,606. The starting compounds may be obtained also by a known procedure, e.g., those described in Hungarian Patent No. 177578, which is incorporated herein by reference, namely by coupling an amidoxime having Formula (1), wherein R¹ and R² of Formula (1) is H, with, e.g., a reactive derivative having Formula (2) in the presence of a base, and may be diazotized usually without isolation or purification.

Alternatively, for compounds in which R⁷ is H and m is 1, the compounds may be prepared by the reaction of an oxyrane of Formula (3) and amine of Formula (4). This procedure also may be used for the preparation of compound in which R⁵ is H.

Alternatively, for compounds in which R is represented by Formula (b) and R⁷ is an acyl group, the compounds may be prepared by the esterification of the corresponding compounds in which R⁷ is H. Alkyl or aryl esters may be obtained with an acid chloride or anhydride in the presence of a tertiary amine or an inorganic base, preferably in an inert solvent, or in certain cases by the Schotten-Bauman procedure using aqueous inorganic base in a two-phase system. For the preparation of the aminoacyl esters, carboxyl-activated N-protected amino acid derivatives (e.g., active esters) may be used as reagents in procedures basically known from the peptide chemistry. This coupling also requires the presence of a base (e.g., triethylamine). The isolation and purification of the products may be performed by using standard preparative techniques; the final preparation may often be in the form of a salt with appropriate inorganic or organic acids. Starting from chiral amino acids, the products may frequently be diastereomers, possessing the second chiral center in the R group. During the isolation, these diastereomers often may separate, and the product may be obtained in stereo-pure form.

In yet another embodiment of compounds of Formula (I), Z is a chemical bond, X is halo; A is a group of the Formula (c) and R is a group of the Formula (d):

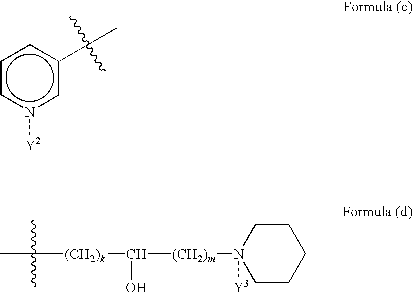

one or both of Y² and Y³ from which at least one must be present in the molecule, are oxygen, or an alkyl or substituted alkyl having 1-4 carbon atoms, k is 1, 2, or 3; and m is 1, 2, or 3. Y² and Y³ are attached by the dotted line. In some aspects of this embodiment, X is chloro or bromo. When the compound is a mono- or bivalent cation, the anion thereof is one or two halide ions. In some aspects of this embodiment, the anion is an iodide ion.

According to this embodiment, the compounds may be prepared by chemical modifications of the terminal pyridine and/or piperidine groups in their unsubstituted precursors, e.g., by N-oxidation or quaternerization. In some aspects of this embodiment, the compounds may be prepared by oxidation with peracids in inert solvents. In further aspects of this embodiment, the peracid is a substituted perbenzoic acid. In further aspects of this embodiment, the inert solvent is chloroform or dichloromethane. If both oxidizable groups are present, mono- or dioxides may form depending on the quantity of the reagent used. At the end of the oxidation reaction, the excess reagent is decomposed and the product is isolated by evaporation. In other aspects of this embodiment, the compounds may prepared by quaternerization. In some aspects of this embodiment, the compounds may be prepared by quarternization with alkyl halides. In some aspects of this embodiment, the alkyl halide is methyliodide. In further aspects of this embodiment, the compound may be prepared by refluxing the reagent in a suitable solvent. In some aspects, the solvent is acetone. In some aspects of this embodiment, the compound is insoluble in the medium, and may be isolated by simple filtration.

In yet another embodiment of compounds of Formula (I), Z is a chemical bond, A is selected from the group consisting of aralkyl, substituted aralkyl, phenyl, substituted phenyl having one or more substituents, a N-containing heteroaryl group, which may be condensed with benzene ring, and a sulfur containing heteroaromatic group; X is —NR¹R², wherein R¹ and R², independently from each other, are selected from the group consisting of H, a straight or branched alkyl a substituted straight or branched alkyl, cycloalkyl and R¹ and R² taken together with the nitrogen atom attached thereto may form a saturated 3 to 7-membered heterocyclic ring; R is a group of the Formula (e)

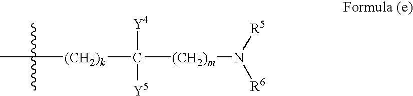

wherein R⁵ and R⁶, independently from each other, are selected from the group consisting of H, a straight or branched alkyl, or a substituted straight or branched alkyl or cycloalkyl, or R⁵ and R⁶ taken together with the nitrogen atom attached thereto form a saturated 3- to 7-membered ring, which may contain additional hetero atoms and substituents; Y⁴ is selected from the group consisting of H, alkyl and substituted alkyl having 1-4 carbon atoms; Y⁵ is selected from the group consisting of H, alkyl and substituted alkyl having 1-4 carbon atoms, or OR⁷, wherein R⁷ is H or an acyl; k is 1, 2, or 3; and m is 1, 2, or 3, with the proviso that when A is phenyl which is unsubstituted or substituted with halogen or alkoxy, or phenylalkyl substituted with alkoxy, or a pyridyl group, and R⁷ is H, then at least one of R¹ and R² is other than H, or when A is phenyl which is unsubstituted or substituted with halogen or alkoxy phenylalkyl substituted with alkoxy, or pyridyl, and R¹ and R² are each H, then R⁷ is other than H.

In some aspects of this embodiment, A is phenylalkyl or phenyl. In some aspects, when A is phenylalkyl, the phenyl may be substituted with one or more alkoxy groups. In some aspects, the alkoxy group has 1 to 4 carbon atoms. In other aspects, A is substituted phenyl having one or more substituents. In some aspects, the substituent groups are selected from the group consisting of an alkyl, preferably alkyl or haloalkyl having 1 to 4 carbon atom, halo, acylamino or nitro group. In other aspects, A is selected from the group consisting of pyrrolyl, pyridyl, isoquinolyl, quinolyl and thienyl. In some aspects, when A is a heteroaryl group, it may be substituted with one or more alkyl, preferably alkyl having 1 to 4 carbon atoms.

In some aspects of this embodiment, R¹ and R², independently from each other, are alkyl having 1 to 6 carbon atoms. In other aspects, when R¹ and R² are taken together with the nitrogen atom attached thereto form a saturated 5-7 membered heterocyclic ring.

In some aspects of this embodiment, R⁵ and R⁶, independently from each other, are alkyl having 1 to 4 carbon atoms. In other aspects, when R⁵ and R⁶ are taken together with the nitrogen atom attached thereto to form a ring, the ring is a 5 to 7 membered saturated heterocyclic ring, which may contain additional hetero atoms and substituents. In this aspect, the substituents may be alkyl having 1 to 4 carbon atoms.

According to this embodiment, compounds wherein X is NH₂ may be prepared, similarly to the above-mentioned procedure, by the reaction of the corresponding compound of Formula (1), wherein R¹ and R² of Formula (1) are H, with a compound of Formula 2. The alkylating agent of Formula 2 may contain hydroxyl and/or amino substituents. The reaction requires the presence of an inorganic or organic base, in a preferable manner alcoholic alcoholate solution is used as medium and base. The compounds may be isolated as a salt with a suitable organic or inorganic acid.

According to this embodiment, compounds wherein R¹ and R², one or both of them are other than H may be prepared by two methods. In the first method, an amidoxime of Formula (1), having the required substituents R¹ and/or R², may be reacted with a reactive compound of Formula (2), similarly to the procedure described in the previous paragraph. The substituted amidoximes of Formula (1), used as starting materials, are known from the literature. See, e.g., Chem. Rev. 62, 155-183 (1962), which is incorporated herein by reference.

In the second method, substitution of the halogen atoms in the compounds of Formula (I), wherein Z is covalent bond and X is halogen, by an amine of Formula (5) may result in similar compounds as well. In the case of derivatives bearing an OH substituent in the R group (Y⁴═OH), this hydroxyl group has to be protected before, and deprotected after the substitution reaction, otherwise formation of the cyclic derivatives of Formula (I′) is favored. For the protection, acetyl type protecting groups, e.g., tetrahydropyranyl group, have proven most satisfactory. The protection may be carried out by the reaction of the unprotected compound with dihydropyrane, followed by the halogen/amine displacement, which usually requires refluxing in a solvent, e.g., in alcohol. The deprotection of the product, finally, may be accomplished by acidic treatment, e.g., by boiling the ethanolic solution in the presence of e.g., p-toluenesulphonic acid.

According to another embodiment, compounds of Formula (I) include those wherein Y⁵ is an acyloxy group. They can be prepared by acylation of the corresponding compound in which Y⁵ is OH, which are either known from the literature (e.g, Hungarian Patent No. 177578) or described in the present invention. The reactions may be accomplished identically to what is described for the analogous halo derivatives, wherein R⁷ is an acyl group.

According to another embodiment, compounds of Formula (I) also include those wherein Z is oxygen or an NR³ group wherein R³ is an unsubstituted or substituted alkyl group; X is —NR¹R², wherein R¹ and R², independently from each other, are selected from the group consisting of hydrogen, unsubstituted or substituted straight or branched alkyl, unsubstituted or substituted aryl, and unsubstituted or substituted aralkyl group, or R¹ and R² are taken together with the nitrogen atom attached thereto to form a 3- to 7-membered saturated heterocyclic ring which optionally contains one or more hetero atoms. According to this embodiment, A is selected from the group consisting of an unsubstituted or substituted alkyl, an unsubstituted or substituted aryl, and unsubstituted or substituted aralkyl group. Further according to this embodiment, R is a group of the Formula (b) wherein R⁵ and R⁶, independently from each other are selected from the group consisting of H, straight or branched alkyl, and cycloalkyl, or R⁵ and R⁶ together with the N-atom attached thereto form a 3- to 7-membered saturated heterocyclic ring. According to this embodiment, Y⁶ is H or —OR⁷, wherein R⁷ is H or acyl, k is 1, 2 or 3 and m is 1, 2 or 3.

In one aspect of this embodiment, R¹ and R², independently from each other, are phenyl. In other aspects, when R¹ and R² are taken together with the nitrogen atom attached thereto to form a ring, the ring is a 5- to 7-membered saturated heterocyclic ring which optionally contains one or more heteroatoms. According to some aspects, A is phenyl or substituted phenyl group. According to some aspects, R⁵ and R⁶, independently from each other, are C₁₋₄ alkyl. Alternatively, according to some aspects, R⁵ and R⁶ together with the nitrogen atom attached thereto, form a 3- to 7-membered ring, the ring is a 5- to 7-membered saturated heterocyclic ring. According to some aspects, R⁷ is unsubstituted or substituted alkylcarbonyl or arylcarbonyl.

According to another embodiment, compounds of Formula (I) also include those wherein Z is oxygen and X is —OR, wherein Q is an unsubstituted or substituted alkyl or unsubstituted or substituted aralkyl group, A is an unsubstituted or substituted alkoxy group or an unsubstituted or substituted aralkyl group and R is a group of the Formula (b), wherein R⁵ and R⁶, independently from each other, are selected from the group consisting of H, straight or branched alkyl, and cycloalkyl, or R⁵ and R⁶, together with the nitrogen atom attached thereto, form a 3 to 7-membered saturated heterocyclic ring, Y⁶ is H or —OR⁷, wherein R⁷ is H or acyl, k is 1, 2 or 3 and m is 1, 2 or 3.

In some aspects of this embodiment, R⁵ and R⁶, independently from each other, are C₁₋₄ alkyl. In other aspects, R⁵ and R⁶, when taken together with the nitrogen atom attached thereto form a saturated 3- to 7-membered heterocyclic ring, preferably the ring is a 5- to 7-membered saturated heterocyclic ring. In some aspects, R⁷ is unsubstituted or substituted alkylcarbonyl or arylcarbonyl.

According to another embodiment, compounds of Formula (I) also include those wherein A is selected from the group consisting of unsubstituted or substituted aryl, N-containing heteroaromatic group and S-containing heteroaromatic group, Z is a chemical bond, X is —OQ wherein Q is C₁₋₄ alkyl and R is a group of the Formula (b), wherein R⁵ and R⁶, independently from each other are selected from the group consisting of H, straight or branched alkyl and cycloalkyl, or R⁵ and R⁶, when taken together with the nitrogen atom attached thereto to form a saturated 3- to 7-membered heterocyclic ring, Y⁶ is H, k is 1, 2 or 3 and m is 1, 2 or 3.

In some aspects of this embodiment, A is phenyl. In other aspects, A is pyridyl. In some aspects of this embodiment, R⁵ and R⁶, independently from each other, are C₁₋₄ alkyl. In other aspects, R⁵ and R⁶ are taken together with the N atom attached thereto to form a 5- to 7-membered heterocyclic ring.

According to this embodiment, these compounds may prepared by the reaction of the corresponding compound of Formula (I) wherein X is halo and the corresponding alcoholates, preferably in an alcohol corresponding to the alcoholate, preferably by refluxing. The reaction mixture may be treated with methods known in the art and the product may be isolated by chromatography or salt-forming.

According to yet another embodiment, compounds of Formula (II) include those wherein X is oxygen, A is selected from the group consisting of C₁₋₂₀ straight or branched alkyl unsubstituted or substituted aryl, unsubstituted or substituted aralkyl, naphthyl and N-containing heteroaromatic group, Z is a chemical bond, R′ is selected from the group consisting of H, C₁₋₄ alkyl and aralkyl, Z is a group of the Formula (b), wherein R⁵ and R⁶ independently from each other, are selected from the group consisting of H, straight or branched alkyl and cycloalkyl, or R⁵ and R⁶ are taken together with the N atom attached thereto to form a 3 to 7-membered heterocyclic ring, Y⁶ is H or —OR⁷, R⁷ is H, k is 1, 2 or 3 and m is 1, 2 or 3, with the proviso that when A is other than alkyl and R″ is H, Y⁶ is H.

In some aspects of this embodiment, A is phenyl or halophenyl. In other aspects, A is pyridyl. In some aspects of this embodiment, R′ is phenylalkyl. In some aspects of this embodiment, R⁵ and R⁶ independently from each other, are C₁₋₄ alkyl. In other aspects, R¹ and R⁶ are taken together with the N atom attached thereto to form a 5- to 7-membered saturated heterocyclic ring.

According to yet another embodiment, compounds of Formula (II) also include those wherein Z is selected from the group consisting of a covalent bond, oxygen and an NR³ group, wherein R³ is hydrogen or an unsubstituted or substituted alkyl group, X is ═NR⁴, wherein R⁴ is selected from the group consisting of hydrogen, an unsubstituted or substituted alkyl, an unsubstituted or substituted aryl, and a substituted or unsubstituted aralkyl. According to this embodiment, A is selected from the group consisting of an unsubstituted or substituted alkyl, an unsubstituted or substituted aryl, an unsubstituted or substituted aralkyl, and cycloalkyl, R′ is selected from the group consisting of an unsubstituted or substituted alkyl, an unsubstituted or substituted aryl, and an unsubstituted or substituted aralkyl, R is a group of the Formula (b), wherein R⁵ and R⁶, independently from each other, are selected from the group consisting of H, straight or branched alkyl or R⁵ and R⁶ are taken together with the N atom attached thereto to form 3- to 7-membered saturated heterocyclic ring, Y⁶ is H or —OR⁷, R⁷ is H or acyl, k is 1, 2 or 3 and m is 1, 2 or 3.

In some aspects of this embodiment, R⁴ is phenyl or phenylalkyl. In some aspects of this embodiment, A is selected from the group consisting of phenyl, substituted phenyl, and phenylalkyl. In some aspects of this embodiment, R′ is phenyl or phenylalkyl. In some aspects of this embodiment, R⁵ and R⁶, independently from each other, are C₁₋₄ alkyl. In other aspects, R⁵ and R⁶ are taken together with the N atom attached thereto to a form 5- to 7-membered saturated heterocyclic ring. In some aspects of this embodiment, R⁷ is unsubstituted or substituted alkylcarbonyl or arylcarbonyl.

According to yet another embodiment, compounds of Formula (II) also include those wherein X is oxygen, A is unsubstituted or substituted alkyl unsubstituted or substituted aralkyl, Z is oxygen, R′ is alkyl or aralkyl, preferably phenylalkyl, R is a group of the Formula (b), wherein R⁵ and R⁶, independently from each other, are selected from the group consisting of H, straight or branched alkyl and cycloalkyl, or R⁵ and R⁶, when taken together with the N atom attached thereto form a 3 to 7-membered saturated heterocyclic ring, Y⁶ is H or —OR⁷, R⁷ is H or acyl, k is 1, 2 or 3 and m is 1, 2 or 3. In some aspects, R⁵ and R⁶, independently from each other, are C₁₋₄ alkyl. In other aspects, R⁵ and R⁶ are taken together with the N atom attached thereto to form a 5- to 7-membered heterocyclic ring. In some aspects, R⁷ is unsubstituted or substituted alkylcarbonyl or arylcarbonyl.

In some aspects of this embodiment, A is phenylalkyl. In some aspects, R′ is phenylalkyl.

According to yet another embodiment, compounds of Formula (II) also include those wherein X is oxygen and Z is ═NH.

According to one embodiment, compounds of Formula (II) include those wherein A is selected from the group consisting of unsubstituted or substituted alkyl, cycloalkyl, and unsubstituted or substituted aralkyl, R is a group of the Formula (b), wherein R⁵ and R⁶, independently from each other, are selected from the group consisting of H, straight or branched alkyl, and cycloalkyl, or R⁵ and R⁶ are taken together with the N atom attached thereto to form a 3- to 7-membered heterocyclic ring, Y⁶ is H or —OH, k is 1, 2 or 3 and m is 1, 2 or 3.

In some aspects of this embodiment, A is phenylalkyl, unsubstituted phenyl or phenyl substituted with halo, alkyl, haloalkyl, alkoxy or nitro. In other aspects, R⁵ and R⁶, independently from each other, are C₁₋₄ alkyl. In other aspects, R⁵ and R⁶ are taken together with the N atom attached thereto to form a 5- to 7-membered heterocyclic ring.

According to one embodiment, compounds of Formula (II) include those wherein A is a group of the Formula (a):

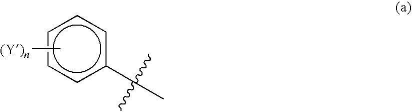

wherein Y¹ is haloalkyl, n is 1, 2 or 3, R′ is H and R is a group of the Formula (b), wherein R⁵ and R⁶, independently from each other, are selected from the group consisting of H, straight or branched alkyl, and cycloalkyl, or R⁵ and R⁶ are taken together with the N atom attached thereto to form a 3- to 7-membered heterocyclic ring, Y⁶ is H or —OH, k is 1, 2 or 3 and m is 1, 2 or 3.

In some aspects of this embodiment, Y¹ is trifluoromethyl. In other aspects, R⁵ and R⁶, independently from each other, are C₁₋₄ alkyl. In other aspects, R⁵ and R⁶ are taken together with the N atom attached thereto to form a 3- to 7-membered heterocyclic ring.

According to one embodiment, compounds of Formula (II) also include the cyclic compounds of the Formula (I″), wherein A is selected from the group consisting of unsubstituted phenyl, phenyl substituted with halo or nitro, and N-containing heteroaryl, R¹ is H and R″ is a terminal amino-alkyl group mono- or disubstituted on the amino group, the alkyl chain of which having 1 to 5 carbon atoms and the amino substituents, independently from each other, may be one or two straight or branched alkyl or cycloalkyl, or the two amino-substituents, together with the N atom adjacent thereto, form a 3- to 7-membered, preferably 5- to 7-membered saturated heterocyclic ring, or a C₁₋₄ alkyl N-quaternary derivative thereof, with the proviso that when A is 3-pyridyl, R″ is not 1-piperidinylmethyl.

The hydroxylamine derivatives described above may be in the form of pharmaceutically acceptable salts, for example hydrochloride, acetate, propionate, pyruvate, oxalate, malate, malonate, succinate, tartarate, citrate, ascorbate, salicylate, and the like. In certain embodiments, the salt is ascorbate, citrate or malate.

Any of the above compounds may be used alone or in combination, optionally in combination with one or more additional therapeutic agents, for the treatment of diabetic wounds. In any compound described above, a moiety that is shown or described as a genus sharing certain chemical characteristics, e.g., alkyl, heteroaryl, halogen, etc., is nevertheless contemplated to be each distinct and separate from other members of that genus.

In other embodiments, the methods of the invention comprise administering one or more hydroxylamine derivatives to a subject suffering from diabetic wound and one or more additional therapeutic agents. In certain embodiments, the additional therapeutic agent is selected from anti-inflammatory agents, anti-pyretic agents, antibiotics, antifungal, antiviral, growth factors, hormones, and neuroprotective agents.

An anti-inflammatory and/or antipyretic agent may be: a non-steroidal anti-inflammatory (NSAID), aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin and zomepirac; arylbutyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid; carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; others such as acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; and pharmaceutically acceptable salts thereof; a steroidal antiinflammatory such as a glucocorticoid; and other analgesics, such as acetaminophen, and opiates.

Steroidal anti-inflammatory therapeutic agents (glucocorticoids) include, but are not limited to, 21-acetoxyprefnenolone, alclometasone, algestone, amicinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisone sodium phosphate, prednisone, prednival, prednylidene, rimexolone, tixocortal, triamcinolone, triamcinolone acetonide, triamcinolone benetonide, triamcinolone hexacetonide, and pharmaceutically acceptable salts thereof. The dosage of analgesic and/or antipyretic such as aspirin, acetaminophen, etc. will be known to those skilled in the art and can be in the range of 80 mg to 250 mg. The dosage of NSAID will be known to those skilled in the art and can be in the range of 80 mg to 500 mg.

In certain other embodiments, an additional therapeutic agent is a antibiotic. Such antibiotics are especially useful when the diabetic wound such as an ulcer is infected, which often occurs. Antibiotics can be of the types such as beta-lactams, cephalosporins, carbacephems, cephamycins, carbapenems, monobactams, quinolones, tetracyclines, aminoglycosides, macrolides, glycopeptides, chloramphenicols, glycylcyclines, licosamides and fluoroquinolones. Examples of antibiotics include amikacin, amoxicillin, ampicillin, axetil, azithromycin, azlocillin, aztreonam, carbenicillin, cefaclor, cefamandole formate sodium, cefazolin, cefepime, cefetamet, cefixime, cefmetazole, cefonicid, cefoperazone, cefotaxime, cefotetan, cefoxitin, cefpodoxime, cefprozil, cefsulodin, ceftazidime, ceftizoxime, ceftriaxone, cefuroxime, cephalexin, cephalothin, chloramphenicol, cinoxacin, ciprofloxacin, clarithromycin, clindamycin, cloxacillin, co-amoxiclavulanate, dicloxacillin, doxycycline, enoxacin, erythromycin, erythromycin estolate, erythromycin ethyl succinate, erythromycin glucoheptonate, erythromycin lactobionate, erythromycin stearate, ethambutol, fleroxacin, gentamicin, imipenem, isoniazid, kanamycin, levofloxacin, lomefloxacin, loracarbef, meropenem methicillin, metronidazole, mezlocillin, minocycline hydrochloride, mupirocin, moxifloxacin hydrochloride, nafcillin, nalidixic acid, netilmicin, nitrofurantoin, norfloxacin, ofloxacin, oxacillin, penicillin G, piperacillin, pyrazinamide, rifabutin, rifampicin, rifampinmetronidazole, rimethoprim-sulfamethoxazole, roxithromycin, streptomycin, sulfamethoxazole, synercid, teicoplanin, telithromycin, tetracycline hydrochloride, ticarcillin, tobramycin, trimethoprim, vancomycin a combination of piperacillin and tazobactam, and derivatives and/or pharmaceutically acceptable salts thereof.

In some embodiments, the additional agent is an antifungal compound, examples of which include poly(hexamethylene biguanide) hydrochloride and chlorhexidines (N,N″-bis(4-chlorophenyl)-3,12-diimino-2,4,11,13-Tetraazatetrade-canediimidamide), which occurs as a free base as well as various pharmaceutically acceptable salts and esters. In some embodiments, the active agent comprises one or more chlorinated phenols, many of which have antimicrobial, antibacterial, antiviral, or antifungal activity, or some combination thereof. Chlorinated phenol compounds which may be used according to the invention include but are not limited to parachlorometaxylenol, dichlorometaxylenol, triclosan (2,4,4′-trichloro-2 hydroxy di-phenyl ether), 2-chlorophenol, 3-chlorophenol, 4-chlorophenol, 2,4-dichlorophenol, 2,4,6-trichlorophenol, 2,3,4,6-tetrachlorophenol, pentachlorophenol, 4-chlororesorcinol, 4,6-dichlororesorcinol, 2,4,6-trichlororesorcinol, alkylchlorophenols (including p-alkyl-o-chlorophenols, o-alkyl-p-chlorophenols, dialkyl-4-chlorophenol, and tri-alkyl-4-chlorophenol), dichloro-m-xylenol, chlorocresol, o-benzyl-p-chlorophenol, 3,4,6-trichlorophenol, 4-chloro-2-phenylphenol, 6-chloro-2-phenylphenol, o-benzyl-p-chlorophenol, and 2,4-dichloro-3,5-diethylphenol. Preferred chlorinated phenols include triclosan and parachlorometaxylenol.

Further, in some embodiments, the additional agent is one or more quaternary ammonium compounds (e.g., monomeric and polymeric quaternary ammonium compounds), many of which have antimicrobial, antibacterial, antiviral, or antifungal activity or some combination of the foregoing activities. Examples of quaternary ammonium compounds include, but are not limited to, benzalkonium chloride, benzethonium chloride, other benzalkonium or benzethonium halides, cetylpyridinium chloride, dequalinium chloride, N-myristyl-N-methylmorpholinium methyl sulfate, poly[N-[3-(dimethylammonio)propyl]-N′-[3-(ethyleneoxyethylene dimethylammonio)propyl]urea dichloride], alpha-4-[1-tris(2-hydroxyethyl)ammonium chloride-2-butenyl]-omega-tris(2-hydroxyethyl)ammonium chloride, alpha-4-[1-tris(2-hydroxyethyl)ammonium chloride-2-butenyl]poly[1-dimethyl ammonium chloride-2-butenyl]-omega-tris(2-hydroxyethyl)ammonium chloride, poly[oxy-ethylene(dimethyliminio)ethylene (dimethyliminio)-ethylene dichloride], ethyl hexadecyl dimethyl ammonium ethyl sulfate, dimethyl ammonium ethyl sulfate, dimethylethylbenzyl ammonium chloride, dimethylbenzyl ammonium chloride, and cetyldimethylethyl ammonium bromide. One preferred quaternary ammonium compound is benzalkonium chloride.

In other embodiments, the additional therapeutic agents are selected from neuroprotectants, because part of pathology of diabetic wound is enervation of tissues. Suitable neuroprotectants include donepezil, memanine, nimodipine, riluzole, rivastigmine, tacrine, TAK147, xaliproden, and mixtures thereof. Nerve growth factor may also be added as an additional therapeutic agent.

In yet other embodiments, the additional therapeutic agents are growth factors, e.g., human platelet-derived growth factor-BB (commercially available as becaplermin, 0.01% gel for the treatment of lower extremity ulcers in type 2 diabetes patients), vascular endothelial growth factors, and granulocyte colony-stimulating factors.

The hydroxylamine derivatives may be provided to an individual by any suitable means, preferably directly (e.g., locally, as by injection to the wound or the surrounding tissue) or systemically (e.g., parenterally or orally). Where the compound is to be provided parenterally, such as by intravenous, subcutaneous, intramuscular, intraorbital, ophthalmic, intraventricular, intracranial, intracapsular, intraspinal, intracisternal, intraperitoneal, buccal, rectal, vaginal, intranasal or by aerosol administration. According to a preferred embodiment, the pharmaceutical compositions of this invention are orally administered. In another preferred embodiment, the pharmaceutical compositions are administered topically as an ointment, a patch/medicated bandage, or by direct injection into the wound.

The amount of both the compound and the additional therapeutic agent that may be combined with the carrier materials to produce a single dosage form will vary depending upon the host treated and the particular mode of administration. Preferably, the compositions of this invention should be formulated so that a dosage of between 0.1-1 g/kg body weight/day, preferably 0.1-300 mg/kg body weight, can be administered. The dose of the compound depends on the condition and the illness of the patient, and the desired daily dose. In human therapy, the oral daily dose is preferably 10-300 mg. These doses are administered in unit dosage forms, which may be divided into 2-3 smaller doses for each day in certain cases, especially in oral treatment.

In the compositions of the present invention, the compounds of this invention may act synergistically in combination with each other and may further act synergistically in the presence of an additional therapeutic agent. Therefore, the amount of compound(s) and additional therapeutic agent(s) in such compositions will be less than that required in a monotherapy utilizing only that therapeutic agent. In such compositions a dosage of between 0.1-1 g/kg bodyweight/day of the additional therapeutic agent can be administered.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The human equivalent of dosage used for mice in the Examples would serve as a credible starting point to adjust the dosage to individual patient's overall physical condition (weight, age etc.) and the status of the wound. The dosage of compound will also depend upon which particular compound is in the composition. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The actual dosage suitable for a subject can easily be determined as a routine practice by one skilled in the art, for example a physician or a veterinarian given a general starting point.

The compound may be delivered hourly, daily, weekly, monthly, yearly (e.g., in a time release form) or as a one-time delivery. The delivery may be continuous delivery for a period of time, e.g., intravenous delivery. In one embodiment of the methods described herein, the therapeutic composition is administered at least once per day. In one embodiment, the therapeutic composition is administered daily. In one embodiment, the therapeutic composition is administered every other day. In one embodiment, the therapeutic composition is administered every 6 to 8 days, or more specifically, weekly.

An embodiment of the method of the present invention is to administer the therapeutic compound described herein in a sustained release form. Such method comprises implanting a sustained-release capsule, a suppository, or a coated implantable medical device so that a therapeutically effective dose of the hydroxylamine derivative is continuously delivered to a subject of such a method. Sustained release may also be achieved using a patch designed and formulated for the purpose. The hydroxylamine derivative may be delivered via a capsule which allows sustained-release of the agent or the peptide over a period of time. Controlled or sustained-release compositions include formulation in lipophilic depots (e.g., fatty acids, waxes, oils). Also comprehended by the invention are particulate compositions coated with polymers (e.g., poloxamers or poloxamines). Sustained release formulae or devices, or any topical formulations, may additionally contain compositions to stabilize the composition or permeate physiological barrier such as skin or mucous membrane. Exemplary additional components may include any physiologically acceptable detergent, or solvent such as, for example, dimethylsulfoxide (DMSO).

Another aspect of the invention provides pharmaceutical compositions comprising a hydroxylamine derivative for the enhancement of diabetic wound healing. Such a composition comprises a hydroxylamine derivative and a pharmaceutically suitable carrier.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; for oral dosage, a syrup, tablet, capsule, gelcap, or palatable solution; for administration by inhalation, a microcrystalline powder or a solution suitable for nebulization; for intravaginal or intrarectal administration, pessaries, suppositories, creams or foams. A preferred formulation is a formulation for oral administration. Another preferred formulation is for topical administration.

Suitable pharmaceutically acceptable carriers that may be used in these pharmaceutical compositions include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, the pharmaceutically acceptable carrier is magnesium stearate. Additional pharmaceutical excipients commonly accepted and used are found in, for example, Remington's Pharmaceutical Sciences (Gennaro, A., ed.), Mack Pub., 1990.

Examples 1-2 below detail compounds of the present invention administered in a diabetic wound animal model. The results showed that iroxanadine and arimoclomol accelerated the healing process of diabetic wound when administered orally or topically. Example 3 below details compounds of the present invention administered in a peripheral nervous system neuropathy animal model, specifically, a diabetic neuropathy animal model in which the diabetic neuropathy is not associated with a diabetic wound. The results showed that iroxanadine provided neuroprotection against the effects of diabetic neuropathy (a neuropathy of the peripheral nervous system).

The foregoing embodiments are presented for illustrative purposes only, and are not intended to be limiting. One of skill in the art will recognize that additional embodiments according to the invention are contemplated as being within the scope of the foregoing generic disclosure, and no disclaimer is in any way intended by the foregoing, non-limiting examples.

The contents of any patents, patent applications, patent publications, or scientific articles referenced anywhere in this application are incorporated herein by reference in their entireties.

### Example 1

Diabetic mice homozygous for the db gene develop insulin-resistant diabetes and obesity due to a defect in the central leptin satiety receptors, essentially eating themselves into diabetes. The db/db mice have previously been shown to undergo delayed wound healing in comparison to non-diabetics.

The BKS.Cg-m^{+/+} Lepr^{db}/J homozygous mouse carries the spontaneous diabetes mutation (Lepr^{db}) and become identifiably obese around 3 to 4 weeks of age. Elevations of plasma insulin begin at 10 to 14 days and of blood sugar at 4 to 8 weeks. Homozygous mutant mice are polyphagic, polydipsic, and polyuric. On the C57BLKS background, these mice exhibit an uncontrolled rise in blood sugar, severe depletion of the insulin-producing beta-cells of the pancreatic islets, and death by 10 months of age. Peripheral neuropathy and myocardial disease are evident, metabolic efficiency is increased, and wound healing is delayed. The BKS.Cg-m^{+/+} Lepr^{db}/J mouse represents a well characterized model of diabetes with characteristic wound healing.

Seventy male BKS.Cg-m^{+/+} Lepr^{db}/J homozygous mice were housed and handled for seven (7) days prior to commencement of the procedure for acclimation purposes. The mice weighed 26.1-41.2 g on Day 0, at the age of 8 weeks. As control, twenty male BKS.Cg-m^{+/-} Lepr^{db}/J heterozygous mice were handled similarly. These mice weighed 19.6-25.1 g on Day 0, at the age of 8 weeks. Mice were divided into Groups (1) to (9), each group consisting of 10 mice.

On Day 0, two full-thickness wounds were made on each mouse in Groups A-I (n=80). Groups (1′) and (2′) consisted of mice that arrived later, and were subjected to the experimental procedure 14 days later, i.e., on Day 14, the same wounding procedure was performed for Groups (1′) and (2′) (the second temporal cohort of db/− controls, n=10).

Wounding Procedure Each mouse was placed into an isoflurane chamber for approximately 5-10 minutes. Each mouse was then shaved, and depilatory cream (Nair) was utilized to remove remaining hair. (Hair removal occurred just before the wounding procedure for Groups (1)-(9) and occurred the day prior to wounding for Groups (1′) and (2′)). The area was cleaned with 70% alcohol and Nolvasan solution. Each mouse was then injected with Buprenorphine and placed back into the isoflurane chamber prior to wound creation. The wound sites were outlined using a 1 cm punch biopsy to ink stamp the mice marking two circular regions on the dorsal left and the dorsal right side. The wound was made by picking up the skin with forceps, and using curved Metzenbaum scissors to make a cut following the template. Buprenorphine was administered at a dose of 0.05 to 0.1 mg/kg SC. The wounds were dressed with Tegaderm dressing, which was changed every 3 days during wound measurement or as necessary.

Immediately following the generation of the skin wound, the original wound was traced by using a fine point maker to outline the perimeter of the wound onto transparency paper (the mouse was immobilized using isoflurane anesthesia for this procedure), and wound size was then traced every 3 days. As the wound neared closure, the assessment of size was conducted every two days and then every day as deemed necessary. Outlines were digitized, quantified and analyzed using computer imaging software as described below.

The mice began daily dosing as detailed in Table 1 on the day of wounding. The mice continued to receive daily treatment until wound closure as determined by the study director and the client. Blood glucose levels were measured by glucometer prior to the first scheduled dosing and weekly thereafter-using blood collected by tail nick. Body weights were measured weekly beginning prior to treatment. Clinical observations were conducted weekly.

TABLE 1
Dosage of test compounds for each group

| | | | | | EtOH | | | | |
|---|---|---|---|---|---|---|---|---|---|
| | | | | Conc. | conc.* | CMC | Vol. | Dose | Dose |
| Group | n | Strain | Treatment | (mg/ml) | (%) | (%) | (ml/kg) | (mg/kg) | Regimen |
| (1), (1′) | 5 | Hetero | Vehicle | n/a | n/a | | 50 | n/a | QD PO |
| control | | | | | | | | | |
| (2), (2′) | 5 | Hetero | iroxanadine | 40 | 37 | 0.6 | 50 | 200 | QD PO |
| (3) | 10 | Homo | Vehicle | n/a | | | 50 | n/a | QD PO |
| control | | | | | | | | | |
| (4) | 10 | Homo | iroxanadine | 2 | 1.9 | 0.98 | 50 | 10 | QD PO |
| (5) | 10 | Homo | iroxanadine | 5 | 4.6 | 0.95 | 50 | 25 | QD PO |
| (6) | 10 | Homo | iroxanadine | 10 | 9.3 | 0.9 | 50 | 50 | QD PO |
| (7) | 10 | Homo | iroxanadine | 20 | 18.5 | 0.8 | 50 | 100 | QD PO |
| (8) | 10 | Homo | iroxanadine | 40 | 37 | 0.6 | 50 | 200 | QD PO |
| (9) | 10 | Homo | arimoclomol | 40 | 0 | 0.63 | 50 | 200 | QD PO |

*For Days 0-8, iroxanadine was dissolved in ethanol, and therefore the formulation also contained ethanol and carboxymethyl cellulose (CMC) in the indicated amounts. For Days 9-12, no ethanol was used, and the amount of CMC was 1.0%. No ethanol was used for arimoclomol.

Upon wound closure, the mice were sacrificed by CO₂ inhalation. Blood was collected by terminal cardiac puncture and processed for serum. Left and right wounds areas were cut out and placed into labeled cassettes and then put into jars of 10% neutral buffered formalin.

The data were collected according to the schedule shown in Table 2.

TABLE 2
Data collection schedule

| Result Type | Collection Time |
|---|---|
| Morbidity and | Daily cage-side observation throughout study |
| Mortality | and clinical observation weekly during dosing |
| Body Weights | Upon study initiation and daily during study |
| Fed Blood Glucose | Weekly |
| Wound Tracing | Every 2-3 Days |
| Blood & Tissue | Upon study completion (wound closure), blood |
| Collection | and wound sites were collected for each mouse |
| Histology | Histology of Wound Tissue |

Raw data was reviewed (read and understood) for accuracy. Wound tracings on transparencies were digitally traced using intuos.3 art pad into Scion Image (Alpha 4.0.3.2). Digital image tracings were analyzed in Scion for perimeter and area and dimensions were standardized to a digital tracing of a reference circle with known dimensions. Percent closure was calculated for both perimeter and area standardized to Day 0 wound size (0% closure). Wound closures on the same mouse had some correlation (Pearson's correlation coefficient=0.71). Therefore, mouse was used as the experimental unit for all analyses except in median time to closure (in which wound was used as the experimental unit).

The ANOVA variance test was used to compare % closure on Day 14, and Student t-tests were used for pair-wise comparisons to db/db vehicle control (Group (3)).

The time to closure using the endpoint of (1) first wound closure on each mouse, or (2) both wound closure on each mouse, was analyzed by survival analysis in JMP 6.0.0 using the proportional hazards model. The proportional hazards model is a special semi-parametric regression model to examine the effect of explanatory variables on survival times. (Cox, D. R., J. Royal Stat. Soc. Ser. B (Methodol.), 1972, 34 (2):187-220). The survival time of each member of a population is assumed to follow its own hazard function.

Proportional hazards model is semi-parametric, meaning it has aspects of being both nonparametric and parametric. It is nonparametric in that it involves an unspecified arbitrary baseline hazard function; however, it is also parametric because it assumes parametric form for the covariates. The baseline hazard function is scaled by a function of the model's (time-independent) covariates to give a general hazard function. Unlike the Kaplan-Meier analysis, proportional hazards computes parameter estimates and standard errors for each covariate. The regression parameters (β) associated with the explanatory variables and their standard errors are estimated using the maximum likelihood method. A conditional risk ratio (or hazard ratio) and its confidence limits are also computed from the parameter estimates.

The survival estimates in proportional hazards are generated using an empirical method (Lawless, J. L., “Statistical Models and Methods for Lifetime Data” John Wiley & Sons, Hoboken, N.J., 1982) and represent the empirical cumulative hazard function estimates, H(t), of the survivor function, S(t), and can be written as S₀=exp(−H(t)), with the hazard function.” (JMP 6.0.0 Help)

According to Spruance et al., Antimicrob. Agents Chemother., 48, 2782-279 (2004), hazard ratios have also been used to describe the outcome of therapeutic trials where the question is to what extent treatment can shorten the duration of the illness. However, the hazard ratio, a type of relative risk, does not always accurately portray how much shorter the duration of an illness may become because of the treatment being examined. To gauge the magnitude of the benefit to the subject, time-based parameters available from the time-to-event curve, such as the ratio of the median times of the placebo and drug groups, is more useful. The hazard ratio is the odds of a patient's healing faster under treatment but does not convey any information about how much faster this event may occur.

Median time to closure for each wound was performed using Analyze-it™ (vsn 1.73). For this analysis, wound was used as the experimental unit. Median time to closure comparisons were made using the non-parametric median test.

Mean time to complete closure for both wounds on each animal was performed using mouse as the experimental unit. Mean time to closure comparisons were made using Student t-tests.

Rate of closure was analyzed for the period in which ethanol (EtOH) was included in the formulation (Days 0-8) as compared to the period immediately following (Days 9-12). Further analysis beyond 12 days would be biased as wounds were beginning to reach full closure beyond 14 days for some mice (decreasing the rate of closure to 0).

The data for two groups of heterozygous mice treated at different times because of the delayed arrival of half of the experimental animals (Groups (1) and (1′), Groups (2) and (2′)) were combined after the conclusion of experiments for both time groups. In the results figures and tables, these groups are simply referred to as Group (1) and Group (2).

There were nine unplanned mice deaths outside of the study protocol allowing 90% of the subjects to be followed to study completion. Eight of the nine deaths occurred in the groups receiving the highest dose (200 mg/kg) of iroxanadine (Group (2) and Group (8)). The clustered occurrence of deaths in the highest dose groups suggest that this dose may be above the maximum tolerated dose (MTD) and approaching LD₅₀. Previously determined acute oral LD₅₀ for iroxanadine in mice is 3800 mg/kg. Furthermore, subchronic studies in the rat indicated that the “no observed adverse effect” level (NOAEL) was 400 mg/kg. This unexpected toxicity apparently associated with the compound may be due to administration with EtOH.

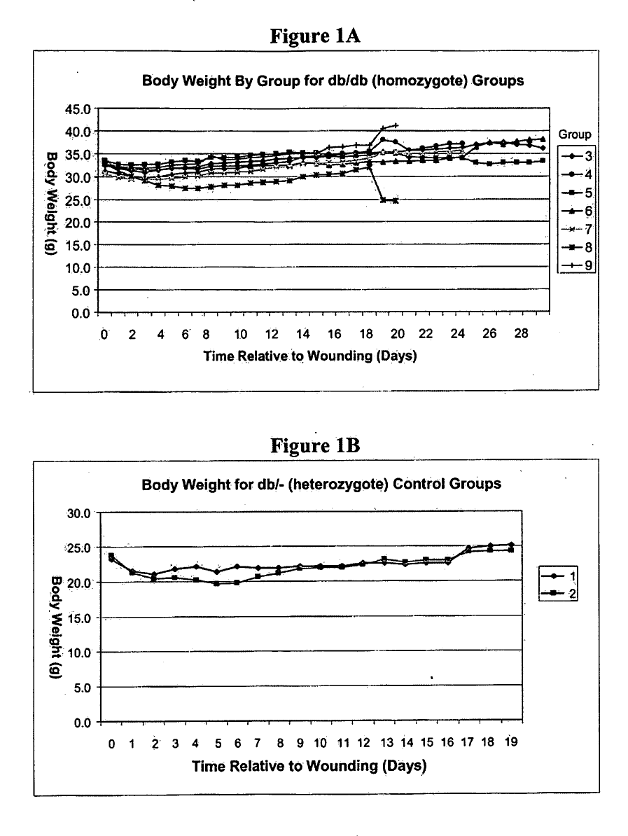

No significant effect on body weight was observed except in diabetic animals treated with the highest dose of iroxanadine. FIG. 1A-B. Body weights were taken daily until wound closure. Sudden changes (increase/decrease) in group averages may be a result of survival bias as a consequence of euthanasia at time of wound closure (reduction in numbers of mice per group).

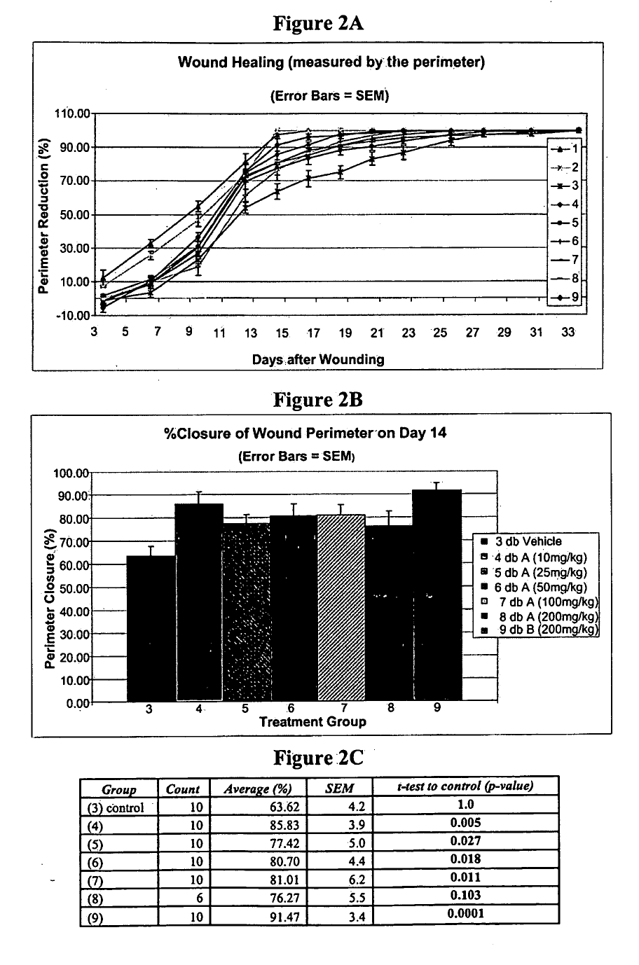

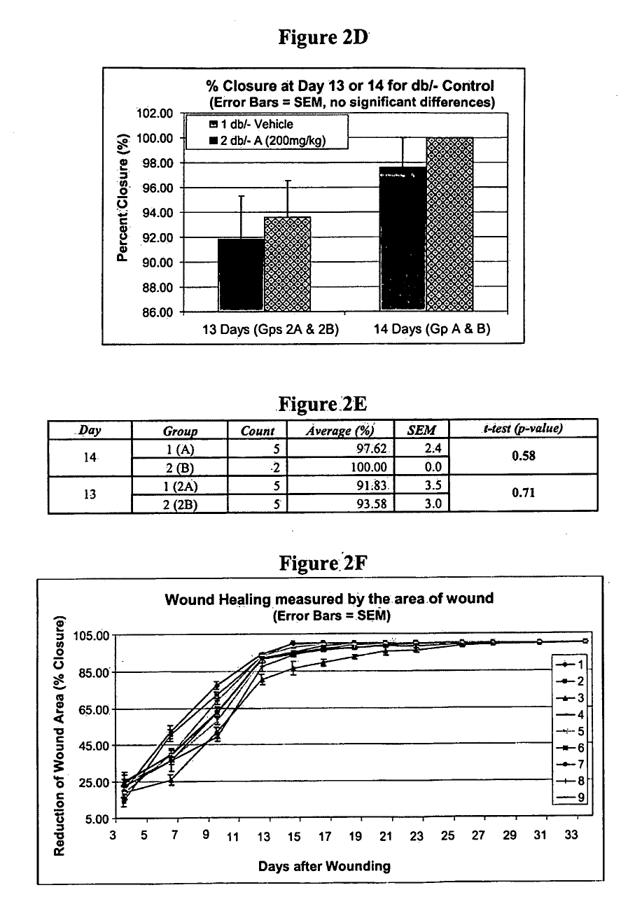

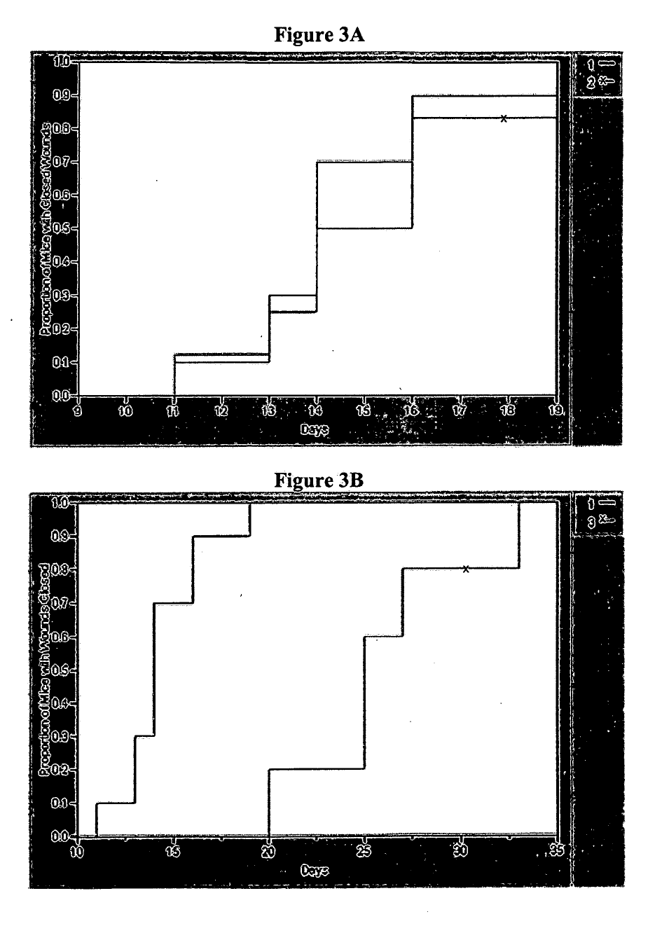

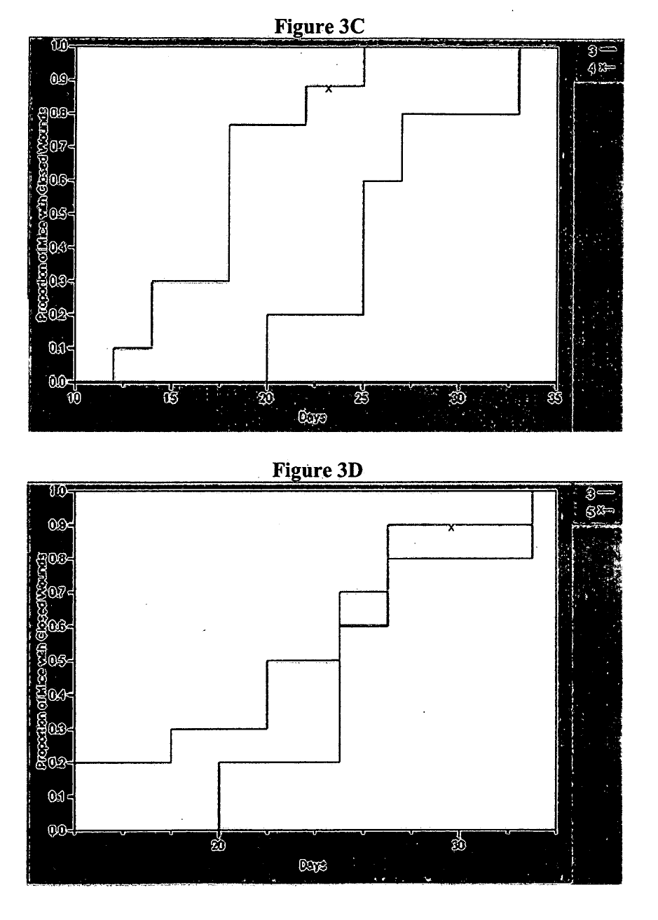

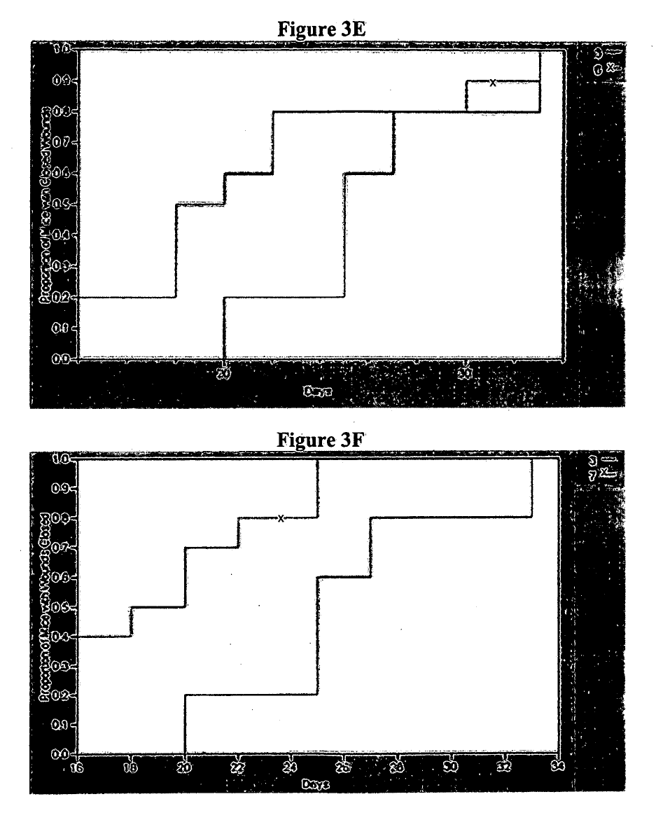

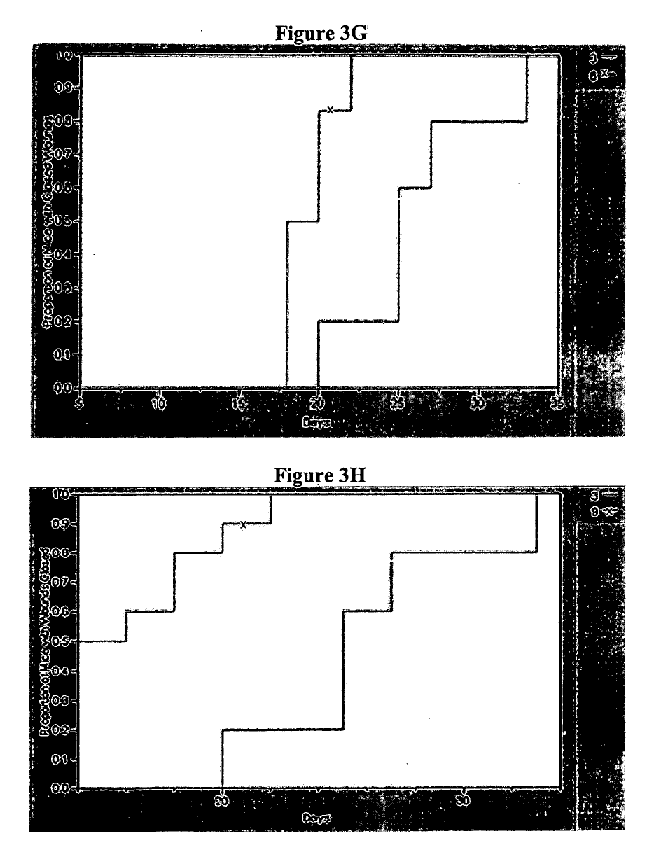

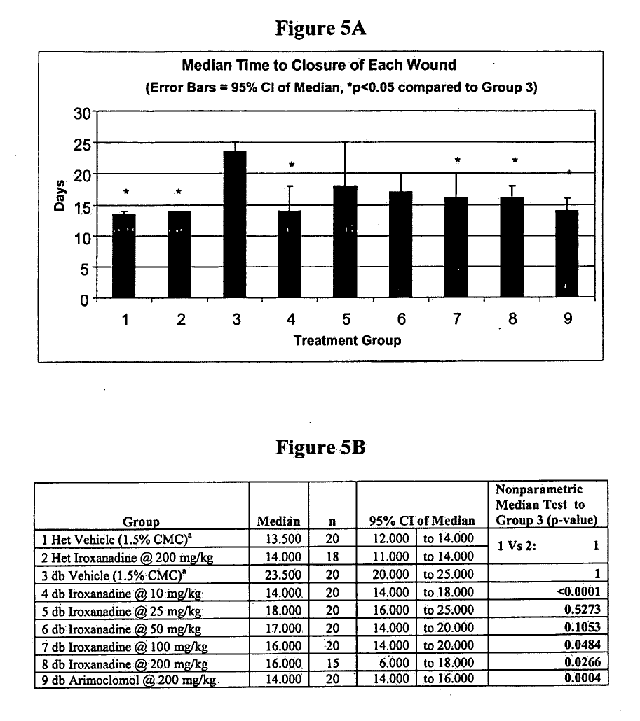

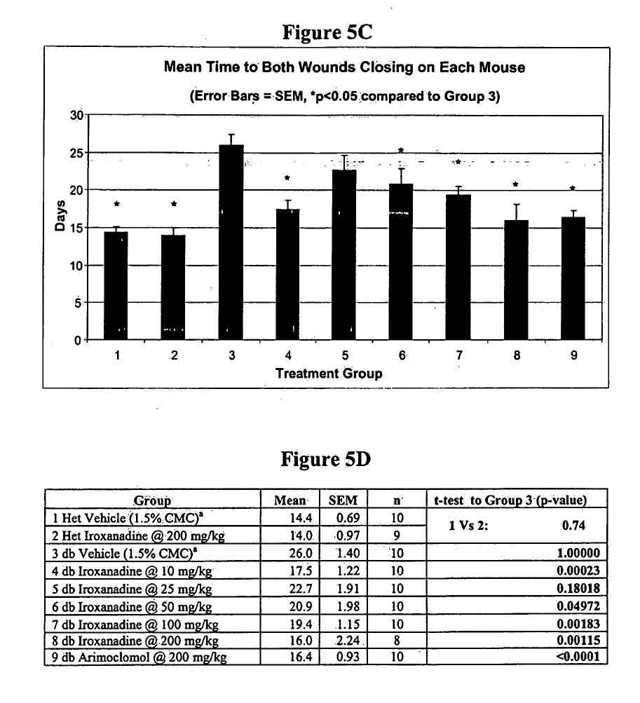

Results of the ANOVA indicated that there were significant differences by treatment group in the percentage closure on Day 14 (FIG. 2B-E), the median time of wound healing (FIG. 5A), and the mean time to complete healing after wounding (FIG. 5B) in diabetic animals, but not in non-diabetic controls. See also wound perimeter (FIG. 2A) or area (FIG. 2F) versus time analysis, which indicates accelerated wound healing in the treatment group.

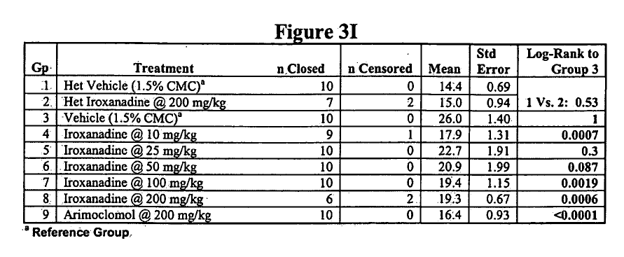

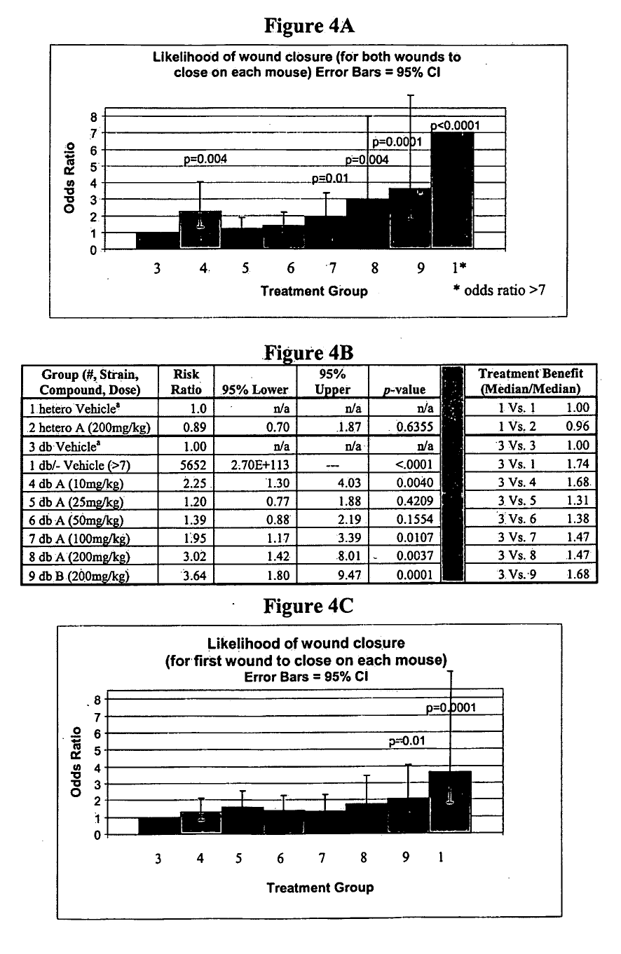

In addition, survival analysis also indicated that there was an increased likelihood of accelerated wound closure based on treatment assignment. (FIG. 4A-C). Panels 4A and 4B show the odds of closure of both wounds, Panel 4A graphically and Panel 4B numerically, and Panel C shows the odds of closure of the first wound, relative to homozygous, diabetic control (Group (3)). The results were analyzed using the proportional hazard model. For closure of both wounds, there was a trend of positive correlation between the odds of closure and iroxanadine administration in a dose dependent manner for diabetic mice (Groups (4)-(8)). Arimoclomol also appeared to be effective in increasing the odds of wound closure (Group 9).

The median time and mean time to closure of the wounds are shown in FIG. 5A-F. Panel 5A graphically and Panel 5B numerically show the median time to closure of each wound singly. Panels 5C and 5D show the mean time for closure of both wounds. The results follow the same trend as the odds of healing, with iroxanadine showing effective shortening of the mean time for wound closing.

Panel 5E shows the rate of wound healing in two periods during the experiment. During the early phase (days 0-8), the test compounds were administered to the mice in ethanol, in effect dosing them in varying amounts of ethanol as well as the test compounds. The last third of the experiment (9-12 days) was carried out with no ethanol. The dose dependent effect was not obvious, but there was a statistically relevant increase in the wound closure rate in groups that were administered either iroxanadine or arimoclomol. These results are also shown in Panel 5F as a box-and-whisker plot. The diamond shape of the left line of each Group shows the mean and the confidence interval around the mean. The lines vertically stretching from the diamond shows the parametric percentile range. The bobbin shape on the right side of each Group shows the median at the notch, the lower and upper quartiles as the top and bottom of the bobbin shape, and confidence interval around the median as the sloped portion. The dotted line connects the nearest observations within 1.5 inter-quartile ranges (IQR) of the lower and upper quartiles. Crosses (+) and small circles (∘) indicate possible outliers, observed at more than 1.5 IQRs (near outliers) and 3.0 IQRs (far outliers) from the quartiles. The vertical lines in the legend show the non-parametric percentile range.

### Example 2

Homozygous, diabetic mice described above, and wounded in the manner described above in Example 1, were treated by topical administration of iroxanadine or arimoclomol. A 4% w/v aqueous solution of arimoclomol were separately administered topically to the wounded position. Oral dosage of iroxanadine was at 10 mg/kg IP, b.i.d., a relatively low concentration.

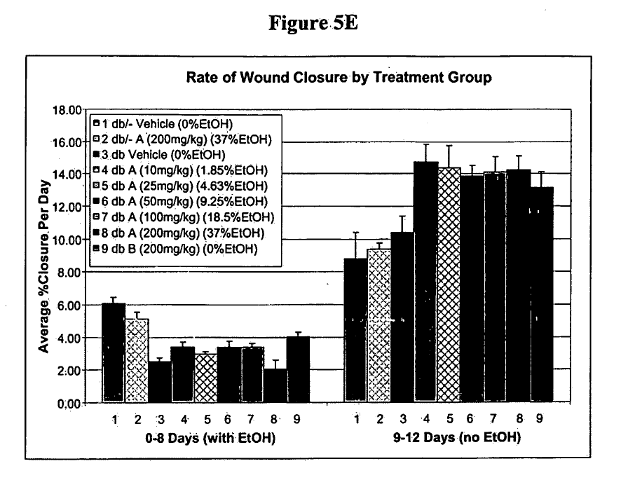

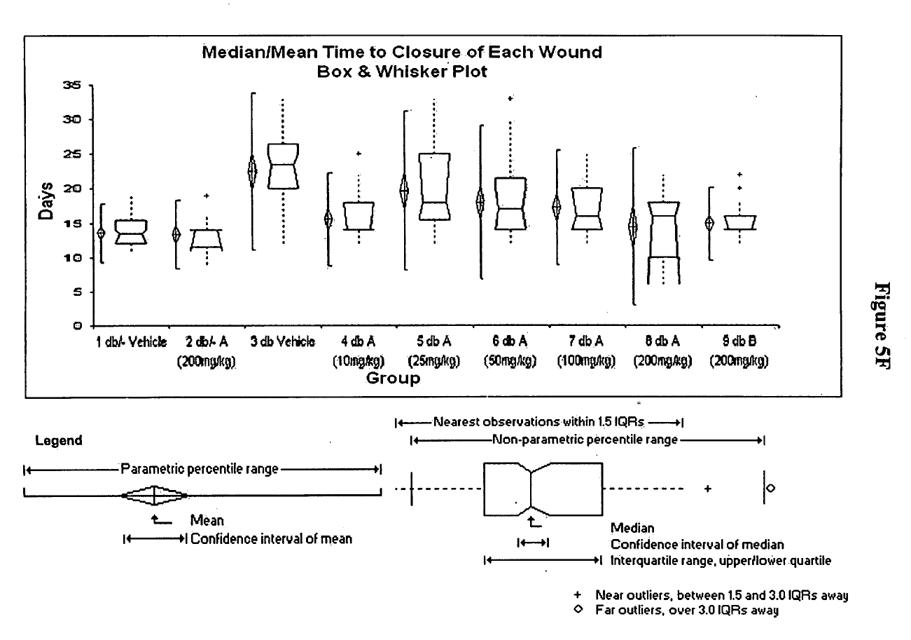

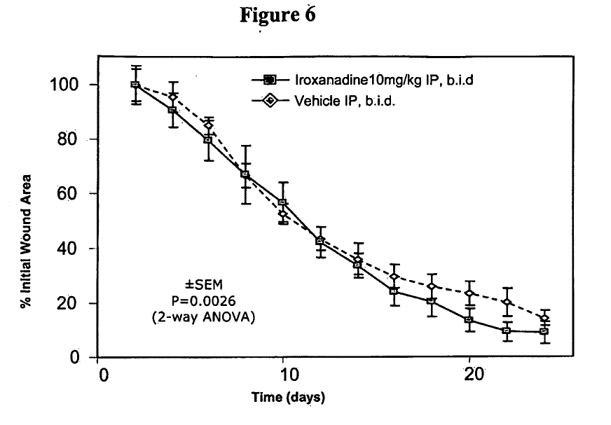

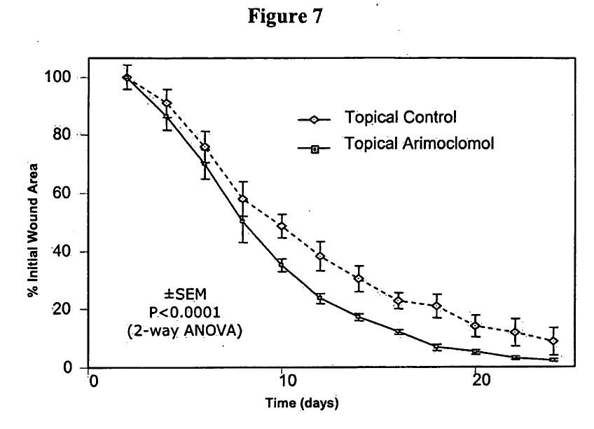

When analyzed by 2-way ANOVA, systemic administration of iroxanadine via oral dosage, after a short delay, tended to show an accelerated healing in the middle stage of healing (FIG. 6). Treatment with arimoclomol clearly showed an accelerated healing of wounds that received topical administration of arimoclomol (FIG. 7).

Thus, there is a statistically significant acceleration of the wound healing process in a subject afflicted with diabetes when a hydroxylamine compound or composition described herein is administered.

Every patent and non-patent publication that is included herein is incorporated herein by reference in its entirety.

The foregoing embodiments are presented for illustrative purposes only, and are not intended to be limiting. One of skill in the art will recognize that additional embodiments according to the invention are contemplated as being within the scope of the foregoing generic disclosure, and no disclaimer is in any way intended by the foregoing, non-limiting examples.

### Example 3

Rat STZ-induced diabetic neuropathy has been described as sharing similar features with human diabetic neuropathy. See Wei et al., Heart Lung Circ., 12:44-50 (2003). Similar to diabetic patients, STZ-rats develop an acute decrease in nerve blood flow and slowing of nerve conduction velocity followed by axonal atrophy of nerve fibers. Nerve fiber degeneration processed as assessed by loss of IENF in skin biopsies of STZ-rats was also demonstrated.

Fifty male Wistar rats were housed and maintained in a room with controlled temperature and a reverse light-dark cycle. Diabetes was induced in forty animals by injection of a buffered solution of STZ (55 mg/kg) in 0.1 mol/citrate buffer pH 4.5. The control group (10 animals) was given an equal does of buffer. The day that STZ was injected was considered day 0. One week later (day 7), blood glucose levels were monitored and rats with glycemia (>260 mg/dl) were deemed diabetic.

The STZ-diabetic rats were divided into four groups. Each group contained ten animals. Iroxanadine was administered to three of the groups in 10, 20 and 50 mg/kg doses, p.o. on a daily basis from day 8 to day 40 post-STZ. The iroxanadine was administered in a 1% carboxymethylcellulose vehicle. The remaining group of 10 STZ-treated animals received vehicle control on the same regimen.

The body weights and glycemia levels of the STZ-diabetic rats were monitored. The animals were also evaluated at days 3, 25-26 and 39-40 post-STZ by measuring compound muscle action potential (CMAP) latency and the loss of sensory nerve conduction velocity (SCNV). These tests were carried out according to Andriambeloson et al., Neuropathology 26:32-42, (2006); and Bordet et al., J Pharmacol Exp Ther 326:623-32 (2008).

For the glycemia level monitoring, the blood glucose levels were measured using a tail incision and a glucosimeter at day 7 and day 41.

For the CMAP latency test, an electrode was inserted into the lower back of the rat, and the ground needle was placed in the hind paw. The right sciatic nerve was stimulated with single electrical pulses (0.2 ms) at a supramaximal intensity (12.8 mA) delivered by a monopolar needle percutaneously placed at the sciatic notch. CMAP was recorded by a needle electrode placed in the gastrocnemius muscle. The latency of CMAP expressed in milliseconds was used as a measure of motor nerve conduction velocity.

For the SNCV measurement, stimulation of the rat caudal nerve was performed at the base of the tail with a pair of needle electrodes. A series of 20 pulses of 0.2 millisecond (ms) duration with an intensity of 12.8 mA were delivered. The evoked response was measured with monopolar needles placed in a proximal site approximately 3-4 cm from the stimulation site. A ground needle electrode was inserted between the stimulating and recording electrode needles. Sensory nerve velocity was expressed in ms according to the distance between the two active electrodes.

On day 41, all animals were anesthetized by intraperitoneal injection of a mixture of 60 mg/kg ketamine and 4 mg/kg xylazine. A 5×10 mm size of skin was punch-biopsied from the hindpaw. Skin samples were immediately fixed overnight in paraformaldehyde at 4° C., incubated overnight in 30% sucrose in 0.1 M PBS (phosphate buffered solution) for cryoprotection, embedded in OCT (Miles, Inc. Diagnostic Division, Elkhart, Ind.) and frozen at −80° C. Cryosections 50 μm-thick were then cut perpendicular to the skin surface with a cryostat. Free-floating sections were incubated for 7 days in a bath of rabbit anti-protein gene product 9.5 (1:10,000; Ultraclone, Isle of Man, UK) at 4° C. The sections were then processed to reveal immunoreactivity by incubating with biotinylated anti-rabbit antibody (1:200) for 1 hour and then for 30 minutes in the avidin biotinylated complex at room temperature. Peroxidase activity was visualized using 3,3′-Diaminobenzidine Tetrahydrochloride. Sections were counterstained with eosin and hematoxylin. Sections were subsequently dehydrated and mounted on Eukitt mounting media (EMS, Hatfield, Pa.). The number of intra-epidermal nerves were counted for the control group and throughout the entire length of the skin section for the STZ-rats using three microscope fields.

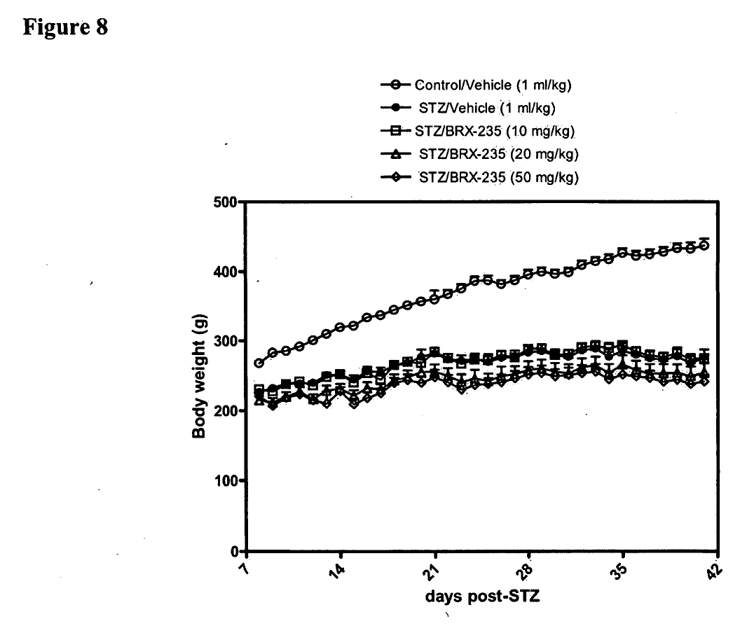

FIG. 8 shows the results of the body weight monitoring of all groups of rats (control, STZ, etc.) from day 7 to day 41. FIG. 8 illustrates that the control (non-STZ) rats continued to gain body weight while the STZ and STZ/iroxanadine treated rats leveled off in body weight after the introduction of the STZ. This indicates that iroxanadine did not improve the over-all diabetic state.

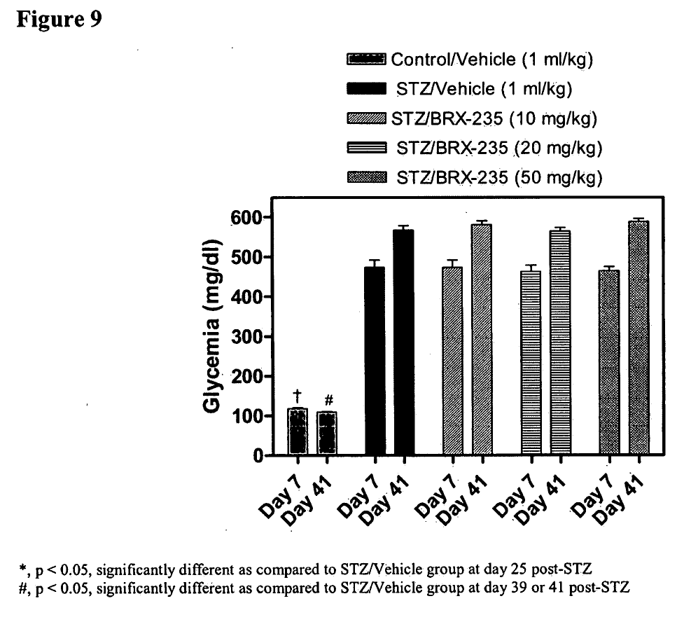

FIG. 9 shows the results of the glycemia level of all groups of rats on day 7 and day 41. FIG. 9 illustrates that the glycemia levels of the iroxanadine treated STZ diabetic rats was lower than non-treated STZ diabetic rats. This indicates that iroxanadine did not improve the over-all diabetic state.

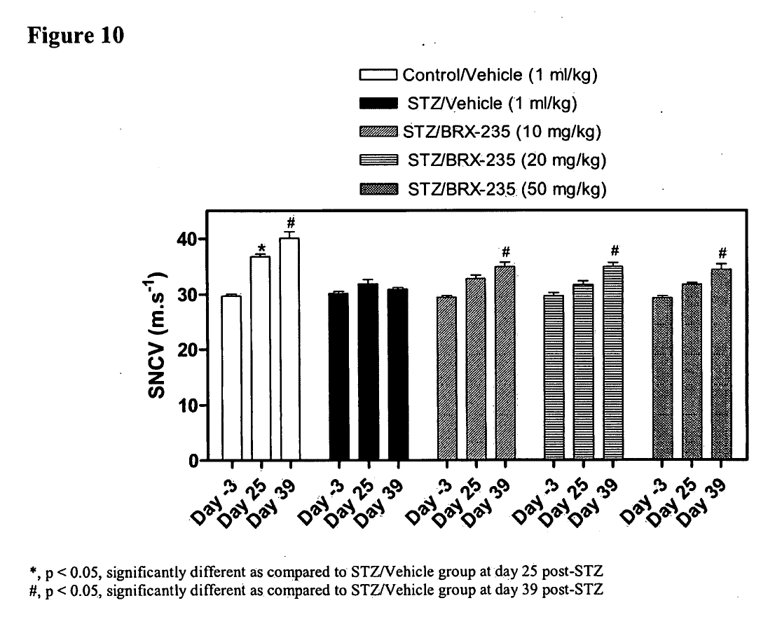

FIG. 10 shows the results of the SNCV performance test of all groups of rats 3 days before STZ-induced diabetes and on days 25 and 39 (post-STZ induced diabetes). FIG. 10 illustrates an increase in nerve conduction velocity of the iroxanadine treated STZ diabetic rats by day 39 compared to the control STZ diabetic rats. This indicates an improvement in peripheral neuropathy.

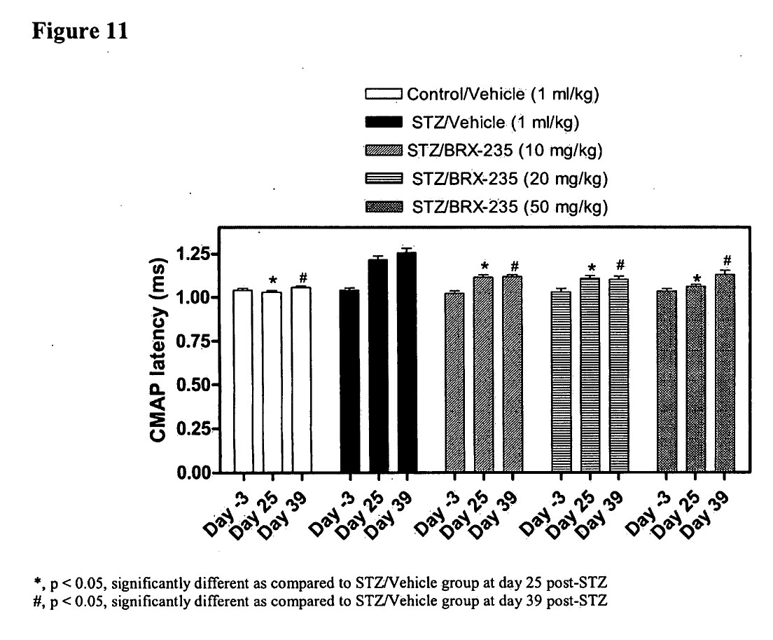

FIG. 11 shows the results of the CMAP latency performance test of all groups of rats 3 days before STZ-induced diabetes and on days 25 and 39 (post-STZ induced diabetes). FIG. 11 illustrates a reduction in the compound muscle action potential latency for each of the iroxanadine treated rat groups compared to the control STZ diabetic rat group, again indicating an improvement in peripheral neuropathy.

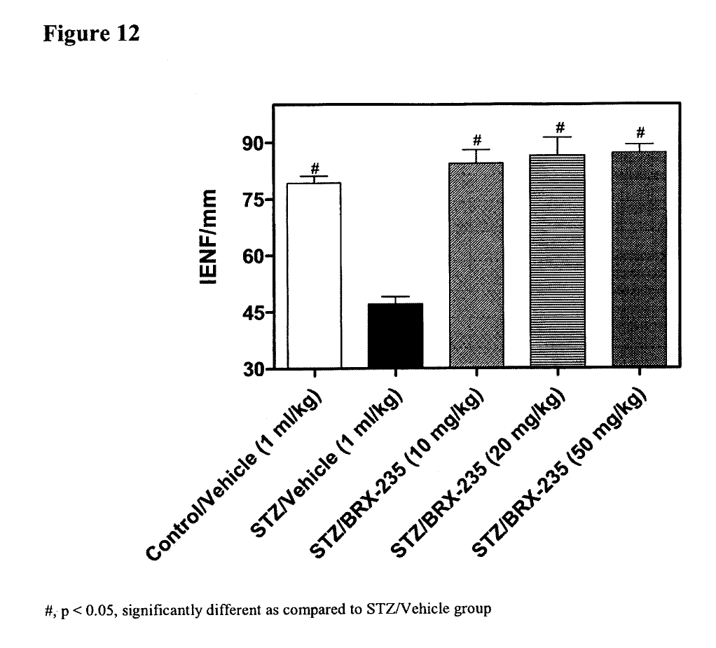

FIG. 12 shows results of the intra-epidermal nerve (IENF) density of the paw skin sample for the post-STZ diabetic rats. FIG. 12 illustrates an increase of IENF density for all iroxanadine treated STZ-diabetic rat groups. In fact, iroxanadine-treated animals had IENF levels that were indistinguishable from that of non-diabetic animals, again indicating an improvement in peripheral neuropathy by iroxanadine treatment.

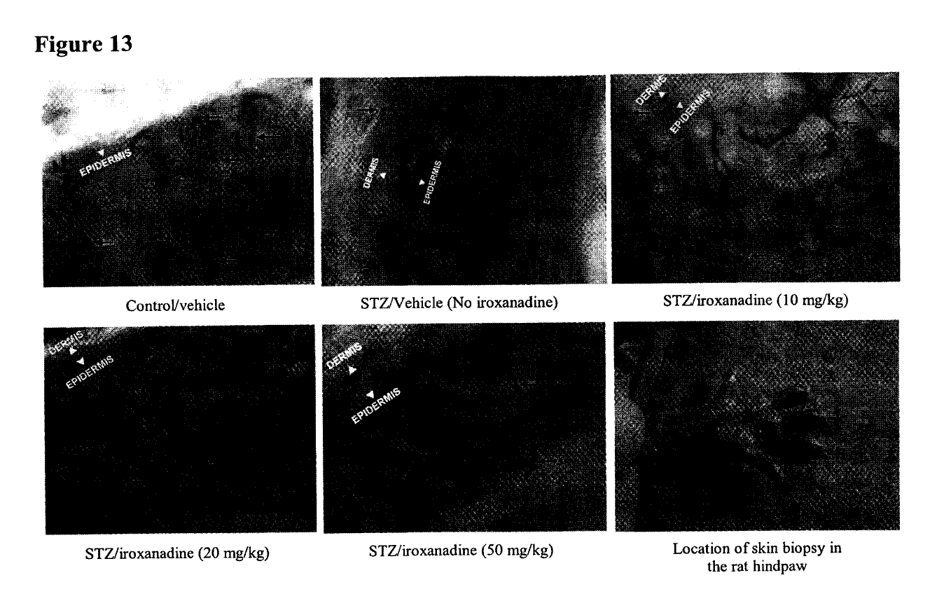

FIG. 13 shows photographs (×100) of the intra-epidermal nerve fibers in each of the experimental rat groups. This illustrates the lack of IENFs in the post-STZ diabetic rats when compared to the iroxanadine treated STZ diabetic rats and the non-treated control rat group. Photomicrographs such as this were used to generate the quantitative data shown in FIG. 13.

In conclusion, it was found that treatment of STZ diabetic rats with iroxanadine improves sensorimotor dysfunction as assessed in the CMAP latency test and the SNCV improvements. Iroxanadine also showed efficacy in preventing the loss of IENF of STZ diabetic rats. These improvements illustrate the potential neuroprotective effects of iroxanadine.

### Example 4

### Treating and Preventing Non-Diabetic Peripheral Neuropathy

Hydroxylamine derivative compounds of the invention are tested in a non-diabetic peripheral neuropathy animal model. See e.g., Ta et al., Molecular Pain, 5:9 (2009); Verdu et al., Muscle Nerve, 22:3, pp. 329-340 (1999). Compounds of Formula I″ are expected to be more effective in ameliorating one or more symptoms of non-diabetic peripheral neuropathy in the animals than are control-treated animals when administered before the neuropathy causing event, supporting the use of such compounds in the prevention of non-diabetic peripheral neuropathies. Such compounds are also expected to be more effective in ameliorating one or more symptoms of non-diabetic peripheral neuropathy in the animals than are control-treated animals when administered before, during or after the neuropathy causing event, supporting the use of such compounds in the treatment or prevention of non-diabetic peripheral neuropathies.

We claim:

1. A method of enhancing healing of a wound associated with diabetes comprising administering to a subject in need thereof an effective amount of a chemical compound, wherein the chemical compound is one or more of a hydroxylamine derivative represented by Formulae (I), (II) or (I″) 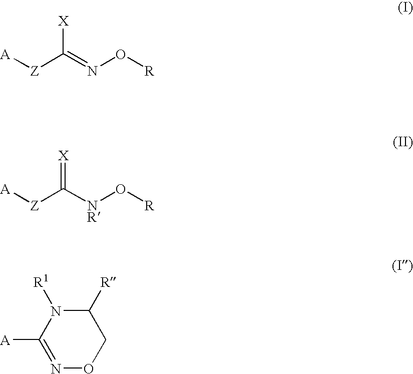
or a salt thereof or any optically active stereoisomer thereof, wherein
A is an alkyl, substituted alkyl, aralkyl, aralkyl substituted in the aryl and/or in the alkyl moiety, aryl, substituted aryl, heteroaryl or substituted heteroaryl group,
Z is a covalent bond, oxygen or NR³ wherein R³ is selected from hydrogen, an alkyl substituted alkyl aryl, substituted aryl, aralkyl and aralkyl substituted in the aryl and/or in the alkyl moiety,
R is alkyl or substituted alkyl,
X of Formula (I) is halo or substituted hydroxy or amino, monosubstituted amino or disubstituted amino group and
X of Formula (II) is oxygen, imino or substituted imino group and
R′ is hydrogen, an alkyl, substituted alkyl, aryl, substituted aryl, aralkyl, aralkyl having substituted aryl or alkyl moiety, acyl or substituted acyl group,
and the compounds of Formula (I) optionally contain intramolecular ring structure of Formula (I″),
thereby enhancing or promoting healing of wound associated with diabetes.

2. The method according to claim 1 wherein the chemical compound is represented by Formula (I), wherein
R is alkyl or substituted alkyl and
(a) Z is covalent bond and X is halogen; or
(b) Z is covalent bond and X is a substituted hydroxy group —OQ, wherein Q is a hydrocarbon; or
(c) Z is covalent bond and X is NR¹R², wherein
R¹ and R², are independently H, linear or branched alkyl, substituted linear or branched alkyl, cycloalkyl, or
R¹ and R², together with the nitrogen atom attached thereto, form a saturated ring containing 3- to 7 members.

3. The method according to claim 2 wherein R is a terminal amino-alkyl optionally substituted on one or more of the amino or alkyl group, and the alkyl chain, which contains 3 to 8 carbon atoms, and is straight or branched, can be substituted with hydroxy or acyloxy.

4. The method according to claim 2 or 3, wherein R is a terminal amino-alkyl mono- or disubstituted on the amino, wherein the amino substituent, independently from each other are one or two straight or branched alkyl or cycloalkyl, or the two amino substituents, together with the nitrogen atom attached thereto form a 3- to 7-membered saturated heterocycle, which may contain additional hetero atom(s).

5. The method according to claim 2, wherein
Z is a covalent bond, and
X is halo, and
A is aralkyl, aralkyl substituted in one or more of the aryl or alkyl moiety, aryl, substituted aryl or heteroaryl.

6. The method according to embodiment 5, wherein A is:
(a) an unsubstituted or substituted phenylalkyl which may have one or more alkoxy substituents,
(b) phenyl,
(c) phenyl substituted with one or more of halo, alkyl, alkoxy, haloalkyl or nitro,
(d) naphthyl,
(e) N-containing heteroaryl which may be condensed with a benzene ring, or
(f) an S-containing or O-containing heteroaryl.

7. The method according to claim 2, wherein
Z is a covalent bond, and
X is a substituted hydroxy of the formula —OQ, wherein Q is straight or branched and wherein Q is unsubstituted or substituted alkyl, unsubstituted aralkyl, or aralkyl substituted in one or more of the aryl or alkyl moiety, and
A is heteroaryl.

8. The method according to claim 2, wherein
Z is a covalent bond,
X is —NR¹R², wherein
R¹ and R² are independently H, a straight or branched unsubstituted alkyl, a substituted straight or branched alkyl, or cycloalkyl, or
R¹ and R², together with the N-atom adjacent thereto, form a 3- to 7-membered saturated ring, and
A is aralkyl, aralkyl substituted in one or both of the aryl or alkyl moiety, unsubstituted or substituted aryl, or heteroaryl.

9. The method according to claim 8 wherein A is
(a) phenylalkyl,
(b) phenylalkyl optionally having one or more substituents in the phenyl moiety,
(c) phenyl,
(d) phenyl substituted with one or more alkyl, halo, alkoxy, haloalkyl, nitro or acylamino,
(e) naphthyl,
(f) N-containing heteroaryl which may be condensed with a benzene ring, or
(g) an S-containing or O-containing heteroaryl.

10. The method according to claim 1 wherein the compound is represented by Formula (I) 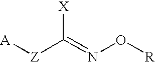
wherein
a) X is halo, Z is chemical bond and
a1) A is a group of the Formula (a), 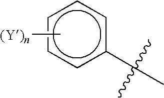
wherein Y¹ is halo, alkoxy, haloalkyl or nitro and n is 1, 2 or 3, or an O-containing heteroaryl, an S-containing heteroaryl, or an N-containing heteroaromatic group which may be condensed with a benzene ring, or the N—C₁₋₄ alkyl quaternary derivative or the N-oxide thereof,
R is a group of the Formula (b), 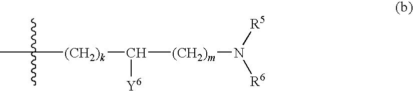
wherein R⁵ and R⁶ are independently H, straight or branched alkyl, or cycloalkyl, or R⁵ and R⁶ when taken together with the N-atom adjacent thereto form a 3- to 7-membered, saturated heterocyclic ring,
Y⁶ is —OR⁷ wherein R⁷ is H or unsubstituted or substituted alkylcarbonyl, arylcarbonyl or aminoacyl, k is 1, 2 or 3, and m is 1, 2 or 3, or an N—C₁₋₄ alkyl-quaternary derivative or N-oxide thereof,
with the proviso that when A is pyridyl, or a group of the Formula (a), wherein Y¹ is halo or alkoxy, R⁷ is other than H, or
a2) A is a group of the Formula (c), 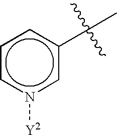
R is a group of the Formula (d), 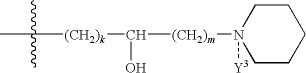
and the optional substituents Y² and Y³, one of which must be present in the molecule, are oxygen or C₁₋₄ alkyl,
k is 1, 2 or 3 and m is 1, 2 or 3 and,
when the compound is a mono- or divalent cation, the anion is one or two halide ion, or
b) A is unsubstituted or substituted aryl or an N-containing heteroaromatic group or an S-containing heteroaromatic group,
Z is chemical bond, X is OQ, wherein Q is C₁₋₄ alkyl, and R is a group of the Formula (b), wherein R⁵ and R⁶ are independently H, straight or branched alkyl or cycloalkyl, or R⁵ and R⁶ when taken together with the N-atom adjacent thereto form a 3- to 7-membered saturated heterocyclic ring,
Y⁶ is H,
k is 1, 2 or 3, and m is 1, 2 or 3.

11. The method according to claim 10 wherein the moiety A is a group of the Formula (a) and Y¹ is C₁₋₄ haloalkyl.

12. The method according to claim 10 wherein the compound is an optically active stereoisomer of a hydroxylamine derivative wherein
X is halo,
Z is chemical bond, and
R is a group of the Formula (b), wherein R⁵ and R⁶ are independently H, straight or branched alkyl or cycloalkyl, or R⁵ and R⁶ when taken together with the N-atom adjacent thereto form a 3- to 7-membered saturated heterocyclic ring,
Y⁶ is —OR⁷ wherein R⁷ is aminoacyl,
k is 1, 2 or 3, and m is 1, 2 or 3.

13. The method according to claim 1 wherein the compound is represented by the Formula (I)
wherein X is NR¹R² wherein R¹ and R² are independently H or unsubstituted or substituted straight or branched alkyl, or cycloalkyl, or R¹ and R², when taken together with the N-atom attached thereto, form a 3- to 7-membered hetero ring,
A is unsubstituted or substituted phenylalkyl substituted with one or more alkoxy, phenyl, phenyl substituted with one or more halo, alkyl or haloalkyl or acylamino or nitro, or an unsubstituted or substituted N-containing heteroaromatic group which may be condensed with a benzene ring, or an S-containing heteroaryl, wherein the hetero atoms may have one or more alkyl substituent(s),
Z is a chemical bond, and
R is a group of the Formula (e), 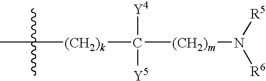
wherein R⁵ and R⁶ are independently H, straight or branched alkyl, or cycloalkyl, or
R⁵ and R⁶ when taken together with the N-atom adjacent thereto form a 3- to 7-membered saturated heterocyclic ring, which may have additional hetero atom(s) and optionally C₁₋₄ alkyl substituent(s),
Y⁴ is H or unsubstituted or substituted C₁₋₄ alkyl,
Y⁵ is H or unsubstituted or substituted C₁₋₄ alkyl or —OR⁷, wherein R⁷ is H or acyl,
k is 1, 2 or 3, and m is 1, 2 or 3,
with the proviso, that when R⁷ is H, at least one of R¹ and R² is other than H, and when R¹ and R² are each H, R⁷ is other than H.

14. The method according to claim 10, wherein in subparagraph (a), A is furyl, thienyl, pyridyl, quinolyl or isoquinolyl.

15. The method according to claim 10, wherein in subparagraph (b), A is phenyl or pyridyl.

16. The method according to claim 13, wherein A is pyrrolyl, pyridyl, isoquinolyl, quinolyl or thienyl.

17. The method according to claim 1, wherein the compound is selected from:
N-[2-hydroxy-3-(1-piperidinyl)propoxy]-3 pyridine-carboximidoyl-chloride (bimoclomol),
N-[2-hydroxy-3-(1-piperidinyl)propoxy]-pyridine-1-oxide-3-carboximidoyl chloride (arimoclomol),
N-[3-(1,1-dimethylethyl)amino]2-hydroxypropoxy]-3-trifluoromethylbenzene-carboximidoyl chloride, and
5,6-dihydro-5(1-piperidinyl)-methyl-3-(3-pyridyl)-4H-1,2,4-oxadiazine (iroxanadine),
or a pharmaceutically acceptable salt thereof or any optically active stereoisomer thereof.

18. The method according to claim 17, wherein the compound is isolated N-[2-hydroxy-3-(1-piperidinyl)propoxy]-pyridine-1-oxide-3-carboximidoyl chloride (arimoclomol) or a pharmaceutically acceptable salt thereof or any optically active stereoisomer thereof.

19. The method according to claim 17, further comprising administering an additional therapeutic agent.

20. The method according to claim 17, further administering a second hydroxylamine derivative.

21. The method according to claim 20, wherein the hydroxylamine derivative is arimoclomol and the second hydroxylamine derivative is iroxanadine.

22. The method according to claim 19, wherein the additional therapeutic agent is selected from anti-inflammatory agents, antibiotics, and neuroprotective agents.

23. The method according to claim 1 wherein the compound is administered systemically.

24. The method according to claim 23, wherein the compound is administered orally.

25. The method according to claim 23, wherein the compound is administered parenterally.

26. The method according to claim 1, wherein the compound is administered topically.

27. The method according to claim 26, wherein the compound is administered via a sustained release device.

28. A device comprising a compound of claim 1 capable of sustained release of said compound for enhancing wound healing in a diabetic subject.

29. The device according to claim 28, wherein the compound is 5,6-dihydro-5(1-piperidinyl)-methyl-3-(3-pyridyl)-4H-1,2,4-oxadiazine.

30. The device according to claim 28, wherein the compound is N-[2-hydroxy-3-(1-piperidinyl)propoxy]-3-pyridine-1-oxide-3-carboximidoyl chloride.

31. The device according to claim 28, wherein the compound is N-[3-(1,1-dimethylethyl)amino-2-hydroxypropoxy]-3-trifluoromethylbenzene-carboximidoyl chloride.

32. A method of treating or preventing a neuropathy of the peripheral nervous system, comprising administering to a subject in need thereof an effective amount of a chemical compound, wherein the chemical compound is one or more of a hydroxylamine derivative represented by Formulas (I), (II) or (I″) 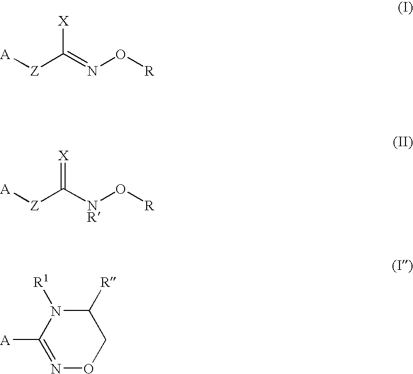
or a salt thereof or any optically active stereoisomer thereof, wherein
A is an alkyl, substituted alkyl, aralkyl, aralkyl substituted in the aryl and/or in the alkyl moiety, aryl, substituted aryl, heteroaryl or substituted heteroaryl group,
Z is a covalent bond, oxygen or NR³ wherein R³ is selected from hydrogen, an alkyl substituted alkyl aryl, substituted aryl, aralkyl and aralkyl substituted in the aryl and/or in the alkyl moiety,
R is alkyl or substituted alkyl,
X of Formula (I) is halo or substituted hydroxy or amino, monosubstituted amino or disubstituted amino group and
X of Formula (II) is oxygen, imino or substituted imino group and
R′ is hydrogen, an alkyl, substituted alkyl, aryl, substituted aryl, aralkyl, aralkyl having substituted aryl or alkyl moiety, acyl or substituted acyl group,
and the compounds of Formula (I) optionally contain intramolecular ring structure of Formula (I″),
with the proviso that:
(a) when the peripheral neuropathy is diabetic neuropathy; and
(b) when the compound is of Formula (I), wherein R is a group of formula (d) 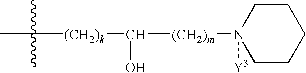
unsubstituted by Y³, X is halo and Z is a chemical bond;
then A cannot be pyridyl or N-oxy pyridyl.

33. A method of treating or preventing a neuropathy of the peripheral nervous system, comprising administering to a subject in need thereof an effective amount of a chemical compound, wherein the chemical compound is one or more of a hydroxylamine derivative represented by Formulas (I), (II) or (I″) 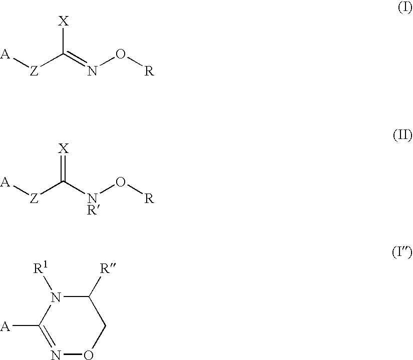
or a salt thereof or any optically active stereoisomer thereof, wherein
A is an alkyl, substituted alkyl, aralkyl, aralkyl substituted in the aryl and/or in the alkyl moiety, aryl, substituted aryl, heteroaryl or substituted heteroaryl group,
Z is a covalent bond, oxygen or NR³ wherein R³ is selected from hydrogen, an alkyl substituted alkyl aryl, substituted aryl, aralkyl and aralkyl substituted in the aryl and/or in the alkyl moiety,
R is alkyl or substituted alkyl,
X of Formula (I) is halo or substituted hydroxy or amino, monosubstituted amino or disubstituted amino group and
X of Formula (II) is oxygen, imino or substituted imino group and
R′ is hydrogen, an alkyl, substituted alkyl, aryl, substituted aryl, aralkyl, aralkyl having substituted aryl or alkyl moiety, acyl or substituted acyl group,
and the compounds of Formula (I) optionally contain intramolecular ring structure of Formula (I″),
with the proviso that:
(a) when the peripheral neuropathy is diabetic neuropathy; and
(b) when the compound is a compound of Formula (I), Z is a chemical bond, X is halo and A is a group of Formula (c),
then R cannot be a group of Formula (d), 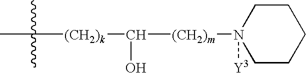
wherein k and m are both 1.

34. The method according to claim 32, wherein the compound is a compound of formula (I″).

35. The method according to claim 34, wherein A is phenyl, substituted phenyl or pyridyl.

36. The method according to claim 35, wherein A is substituted phenyl containing one or more alkyl, halogen, haloalkyl, alkoxy, amino or nitro groups.

37. The method according to claim 34, wherein R″ is a terminal amino-alkyl containing a mono- or di-substituted amino moiety, a terminal amino-alkyl containing a mono- or di-substituted amino moiety and a substituted alkyl moiety.

38. The method according to claim 37, wherein R″ is a terminal C₃₋₈ amino alkyl moiety containing a mono- or di-substituted amino moiety.

39. The method according to claim 38, wherein R″ is a terminal C₃₋₈ amino alkyl moiety, wherein the amino substituents, together with the nitrogen atom attached thereto, form a saturated 3- to 7-membered heterocyclic ring.

40. The method according to claim 39, wherein R″ is a terminal C₃₋₈ amino alkyl moiety, where the amino substituents, together with the nitrogen atom attached thereto, form a saturated 5- to 7-membered heterocyclic ring.

41. The method according to claim 34, wherein the compound is 5,6-dihydro-5(1-piperidinyl)-methyl-3-(3-pyridyl)-4H-1,2,4-oxadiazine (iroxanadine), or a pharmaceutically acceptable salt thereof or any optically active stereoisomer thereof.

42. The method according to claim 34, wherein the compound is administered orally.

43. The method according to claim 34, wherein the compound is administered parenterally.

44. The method according to claim 43, wherein the compound is administered via a sustained release device.

45. The method according to claim 34, wherein the compound is administered topically.

46. The method according to claim 34, wherein the neuropathy is a diabetic neuropathy.

47. The method according to claim 46, wherein the diabetic neuropathy is associated with a diabetic wound.

48. The method according to claim 46, wherein the diabetic neuropathy is not associated with a diabetic wound.

49. A device comprising a compound of claim 32 capable of sustained release of said compound for treating diabetic neuropathy independent of a diabetic wound.

50. The device according to claims 49, wherein the compound is iroxanadine.

51. A method according to claim 32, wherein the neuropathy of the peripheral nervous system is not a diabetic neuropathy.

52. The method according to claim 51, wherein the neuropathy is associated with chemotherapy.
